(19) United States
(12) Patent Application Publication
(10) Pub. No.: US 2001/0055786 A1
(43) Pub. Date: Dec. 27, 2001

(54) Screening method for the discovery and directed evolution of oxygenase enzymes

(75) Inventors: Frances H. Arnold, Pasadena, CA (US); John Joern, Pasadena, CA (US); Takeshi Sakamoto, Machidashi (JP); Ulrich Schwaneberg, Pasadena, CA (US)

Correspondence Address: DARBY & DARBY, 805 THIRD AVENUE, 27TH FLR., NEW YORK, NY, 10022, US

(73) Assignee: CALIFORNIA INSTITUTE OF TECHNOLOGY

(21) Appl. No.: 09/828,599

(22) Filed: Apr. 5, 2001

Related U.S. Application Data
(60) Provisional application No. 60/194,992, filed on Apr. 5, 2000.

(51) Int. Cl. C12Q 1/26

(52) U.S. Cl. 435/25

(57) ABSTRACT

The present invention relates to screening methods for oxidation enzymes, particularly mono- and dioxygenases. According to the methods of the invention, a product of an oxidation reaction is converted into a phenol or a catechol, which is easily detected by a Gibbs assay. This conversion allows for a sensitive and efficient assay. Both high-throughput liquid-phase and solid-phase methods using these principles are provided. Also described are method for detecting phenolic ether-products and sulfhydryl products from oxidation reactions, also using a Gibbs assay.

This application claims priority from U.S. application Ser. No. 60/194,992, filed on Apr. 5, 2000.

The Government has certain rights to this invention pursuant to Grant No. BES-9981770, awarded by the National Science Foundation.

### FIELD OF THE INVENTION

This invention relates to a method for screening enzymes and organisms that will catalyze the hydroxylation of a variety of aromatic substrates.

### BACKGROUND

The publications and reference materials noted herein and listed in the appended Bibliography are each incorporated by reference in their entirety.

Enzymes that are capable of catalyzing the insertion of oxygen into aromatic and aliphatic substrates have many potential applications in pharmaceuticals manufacturing, production of chemicals and also in medicine. According to Faber (1997), “enzymatic oxygenation reactions are particularly intriguing since directed oxyfunctionalization of unactivated organic substrates remains a largely unresolved challenge to synthetic chemistry. This is particularly true for those cases where regio- or enantiospecificity is desired.” Regiospecific hydroxylation of aromatic compounds by purely chemical methods is notoriously difficult. There are reagents for o- and p-hydroxylation available, but some of them are explosive and by-products are usually obtained.

Whereas enzymes such as C. fumago chloroperoxidase (CPO) and various other mono- and dioxygenases such as cytochrome P450 enzymes; methane monooxygenase (MMO) and other di-iron oxygenases; and toluene dioxygenase, have demonstrated the ability to catalyze such reactions, as well as many other interesting and useful oxygen insertion reactions, they are often unsuitable for industrial application due to their low stability and productivity under industrial conditions (e.g., in the presence of organic solvents, high concentrations of reactants, etc.) and, to some extent, their limited substrate ranges. Thus, there is a strong demand for new and improved oxidation catalysts.

Only a very small fraction of enzyme activities existing in nature have been discovered and characterized. Thus, useful oxidation catalysts could be discovered in natural samples by screening natural organisms or expressed gene libraries (Short, 1997). However, an alternative route to finding oxidation enzymes with favorable properties is through directed evolution.

### Directed Evolution

The limitations of naturally occurring enzymes reflect the fact that these enzymes have evolved within the constraints and context of a living organism to carry out specific biological functions, rather than limitations intrinsic to the proteinaceous nature of the catalysts. In some cases, evolution may even specifically require less than optimal activities towards particular substrates. For example, for detoxification enzymes such as P450s, maximizing the flux of substrates to products is unlikely to be the best evolutionary strategy when there is a time-dependent xenobiotic profile (Cook and Atkins, 1997). Therefore, it is highly likely these natural catalysts can be evolved in vitro to be much more efficient industrial oxidative catalysts. Directed evolution is a tool that will be used extensively to alter the properties of oxidative enzymes that use molecular oxygen and their protein cofactors. It will be useful to alter, among other properties, the reactivities, selectivities and stabilities of these enzymes. Furthermore, their selectivity and substrate specificity could be altered to better match the needs of the synthetic chemist. Of particular interest for directed evolution are dioxygenase enzymes, due to their applicability to both industrial chemical syntheses and degradation of environmentally deleterious compounds.

### Dioxygenase Enzymes

Dioxygenase enzymes perform a unique reaction—the insertion of both atoms of molecular oxygen into aromatic substrates to form an enantiomerically pure cis-dihydrodiol (shown below). This reaction was discovered by Gibson et al. (1970) while studying a strain of Pseudomonas putida that utilized toluene as a sole carbon source.

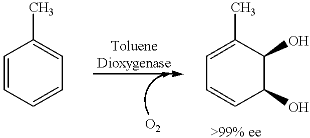

Dioxygenases are also known to catalyze monooxygenation (Pieper et al. (1997); Wackett et al. (1997); Gibson et al. (1989); Gibson and Resnick (1996:1); and Gibson et al. (199)), oxidative dealkylation (Pieper et al. (1997); and Wackett et al. (1997)), sulfoxidation (Gibson and Resnick (1996:3)), desaturation (Gibson and Resnick (1996:3)), and oxidative dehalogenation (Wackett et al. (1997)). In general, these cytochrome 450-type reactions occur with highly substituted aromatic substrates, bicyclic substrates, and highly substituted aliphatic substrates. Diols are often produced along with monols in the case of monooxygenation (Wackett et al. (1997); and Gibson and Resnick (1996:1).

Screening libraries of dioxygenase enzymes created through random mutagenesis and/or recombination using the methods of the invention can provide enzymes with increased practical value, both in the area of bioremediation, and in the synthesis of valuable chiral drugs and specialty chemicals from cis-dihydrodiols, as described below.

Application to Microbial Degradation

According to the Environmental Protection Agency, several substrates of dioxygenase enzymes (e.g., PCB's, ethylene dibromide, dioxin) are priority pollutants, making these enzymes candidates for either bioremediation or waste treatment (World Wide Web address www.epa.gov). Many dioxygenase substrates are highly-substituted aromatics (e.g. tetrachlorobenzene (Pieper et al. (1998)), and hexachlorobiphenyl (Mondello et al. (1997))) which are virtually indestructible in the environment.

Microbial degradation of aromatic compounds by dioxygenase enzymes occurs through a multicomponent enzyme pathway. Understanding this pathway is crucial in determining which enzyme to modify for creation of a library to screen. FIG. 1 shows this pathway for a generic, monosubstituted benzene being degraded by the toluene dioxygenase system.

As shown in FIG. 1, three proteins, including two electron transfer proteins, are required for the first reaction of the TDO degradation pathway. Reductase is a 46 kDa flavoprotein that accepts two electrons from one NADH molecule and shuttles them singly to a 15.3 kDa ferredoxin (Gibson and Zylstra (1989); and Cammack and Mason (1992)). Ferredoxin transfers one electron to the Reiske [2Fe-2S] center of the iron-sulfur protein (ISP), also known as the terminal dioxygenase (Gibson and Zylstra (1989)). The ISP contains both a Reiske [2Fe-2S] center and a conjugated mononuclear iron atom, which is the active site for catalysis. Two electrons are used to charge mononuclear iron of the terminal dioxygenase. At the active site, molecular oxygen is activated, as shown in the postulated mechanism in FIG. 2 (Gibson and Resnick (1996:3)). As the oxygenated species is highly reactive, it must be constrained to the active site to prevent damage to the cell (Mason and Butler (1997)).

The mechanism above predicts a cyclic ester intermediate formed upon substrate binding, resembling the transition state formed upon reaction of the chemical catalyst OsO₄ with arene functionalities to form cis-diols (Gibson and Resnick (1996:3)). However, there is little experimental evidence to support this mechanism, as spectroscopy is only marginally useful in evaluating Fe(II) mononuclear iron centers. The mechanism is based primarily on the similarity between the dioxygenase reaction and the inorganic analog, which both lead to cis-dihydrodiol products.

The activated iron species shown in FIG. 2 is at a high enough oxidation level to catalyze an array of diverse oxidation reactions. After dihydroxylation, the next step in aromatic degradation is formation of a catechol by cis-dihydrodiol dehydrogenase, followed by oxidative cleavage of the catechol by catechol 2,3-dioxygenase (FIG. 1). The final product of the degradation pathway is utilized by the tricarboxylic acid cycle (Hudlicky et al. (1996:2)). All dioxygenase systems have analogs of the five enzymes in FIG. 1. The electron transfer proteins have been shown to be essential for the initial dioxygenation. To produce cis-dihydrodiol, the genes for cis-dihydrodiol dehydrogenase and catechol 2,3-dioxygenase are deleted from the dioxygenase cistron.

Applications to Chemical Syntheses

The potential of cis-dihydrodiols for derivatization through arene functionalities makes them valuable synthetic building blocks for chiral drugs and specialty chemicals. However, the chemistry shown above, i.e., the conversion of toluene to enantiomerically pure cis-dihydrodiol, is virtually impossible to accomplish using conventional chemical synthesis. Conventional oxygenation reactions typically require high temperature and pressure, and produce an array of byproducts that must be separated and destroyed. By contrast, the dioxygenase reaction occurs at room temperature and produces one product with greater than 99% enantiomeric excess.

The biological reaction of aromatics to cis-dihydrodiols has no simple chemical counterpart. For most substrates, dioxygenase enzymes form cis-dihydrodiols with greater than 97% enantiomeric excess. Described below are the synthesis of specific molecules from cis-dihydrodiols, and general schemes for derivatization to more complex molecules.

Synthetic pathways from cis-dihydrodiols usually start by protection of the cis-dihydrodiol as an acetonide, as shown below (Hudlicky et al. (1996:2)).

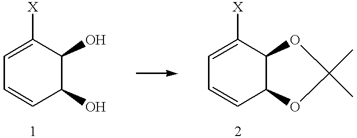

This protection allows for substitution reactions at ring positions 4,5, and 6 (relative to substituent X). The acetonide is removed readily with TFA (Hudlicky et al. (1996:2)). A similar mechanism can be used to attach cis-dihydrodiols to a resin, as shown below (Wendeborn 1998)).

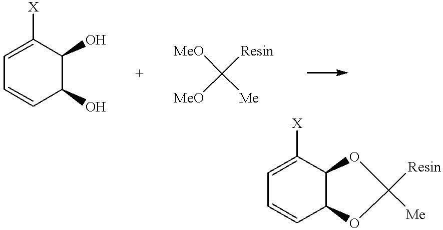

The solid-supported diols can be derivatized by epoxidation, nucleophilic ring opening, Pd(0) cross-linking, and Diels-Alder reactions. Wendeborn (1998) has produced sixteen complex compounds stereoselectively in this manner. Products were removed from the support using TFA.

When substituent X in 1 is a halogen, useful syntheses have been proposed that incorporate either dehalogenation at some later point in the synthesis or substitution of other functional groups at the halogenated position (Hudlicky et al. (1996:1); Hudlicky et al. (1996:2); Sheldrake (1992); and Boyd et al. (1991). In the former scenario, the halogen is a sacrificial way to introduce chirality into the molecule.

In a study by Boyd et al. (1991), eight novel cis-dihydrodiols were created by derivatizing halogenated cis-dihydrodiols by substitution reactions involving halogen removal. Chemical conversion of these halogenated cis-dihydrodiols gave reasonable yields in most cases. This is an alternate pathway to obtaining cis-dihydrodiols with large substituents which are difficult to obtain directly through a biotransformation because of steric considerations. Using this method, phenylacetylene cis-dihydrodiol was synthesized from iodobenzene, as shown below. Direct biotransformation of phenylacetylene gave <5% yield.

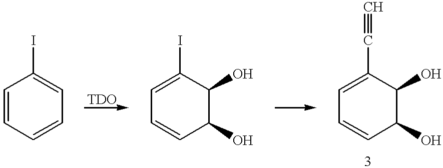

Compound 3 is a precursor to m-hydroxyphenolacetylene, which is a component of a high-performance resin. Treatment of 3 with K₂CO₃ and MeOH for long times yielded exclusively the desired meta isomer. Dehydration of 3 by acidification gave the ortho isomer, but the meta isomer was accessible by dehydration of dioxolane cis-dihydrodiols, followed by incorporation of the acetylene functionality (Wackett et al. (1990)).

Another example of the use of halogenated cis-dihydrodiols is the synthesis of pancratistatin, an alkaloid anti-tumor agent, as described by Hudlicky et al. (1996:1). TDO was used to transform either bromobenzene or chlorobenzene to the corresponding cis-dihydrodiol. After the diol was protected by formation of acetonide 4, the halogen directed the addition of nitrogen to form vinylaziridine 5, as shown below. Pancratistatin was then synthesized in 12 steps from compound 5 with only 2% yield.

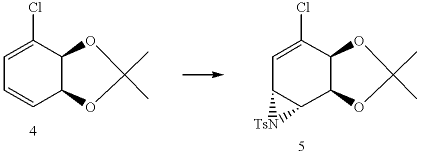

Halogenated cis-dihydrodiols also facilitate the production of conduritols, a family of glycosidase enzyme inhibitors (Sheldrake et al. (1992)).

Another class of syntheses utilizes the dioxygenation of bicyclic compounds. Two examples are indigo synthesis (Genencor International) (Ensley (1994)) and indinavir production (Merck & Co.) (Reider (1997)). Indigo has been synthesized chemically since the late 1800's (Frost and Lievense (1994)). In the Genencor process, D-glucose was converted via 11 separate enzymatic reactions to indole, which was converted by naphthalene dioxygenase to the corresponding cis-dihydrodiol, which spontaneously formed indigo.

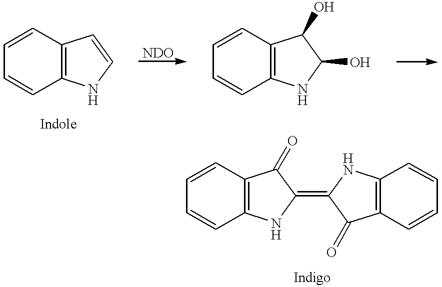

The production of indinavir, the active ingredient of Crixivan, an important HIV protease inhibitor, is another example of a stereoselective synthesis using a dioxygenase to produce a starting material (Drew et al (1999)). Indinavir has five chiral centers, necessitating a complex, multi-step mechanism for its synthesis (Reider et al. (1997)). One of the building blocks of Indinavir can be synthesized by dioxygenation of indene by toluene dioxygenase, followed by chemical conversion to compound 9, cis-1S-amino-2R-hydroxyindan.

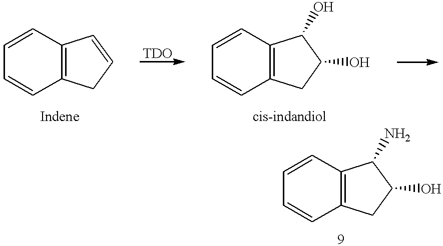

Compound 9 was produced by Reider et al. (1997) with 99% ee via the enzymatic route; using a chiral chemical catalyst results in only 87% ee.

### Limitations of the Applicability of Dioxygenases

Unfortunately, many of the syntheses described in the previous section are prohibitively expensive due to the cost of producing the required cis-dihydrodiols. Dioxygenase enzymes have substrates that are both insoluble in water and toxic to cells. They require an expensive cofactor and enzymes dedicated to electron transfer. They are inhibited by their products. They are unstable at room temperature and have low activity toward unnatural substrates. In short, they are difficult catalysts to use. This section will discuss these highly interrelated shortcomings, and how they can be overcome through both directed evolution and optimization of reaction conditions.

Activity

In general, dioxygenase enzymes show respectable biotransformation rates on their natural substrates. When toluene dioxygenase was expressed in Psuedomonas putida strain NG1, toluene cis-dihydrodiol was produced at a maximum rate of 1.6 g/h/g cell carbon, and accumulated to concentrations as high as 24 g/L (Jenkins et al. (1986)). Though this level of activity may be adequate for an application, other substrates that are more desirable as chiral precursors are not as readily accepted by dioxygenase. For example, toluene dioxygenase is five times less active toward chlorobenzene than toluene, eight times less active toward trichloroethylene, and twenty times less active toward trans-dibromoethylene (Wackett et al (1997)). Bioconversion of halogenated benzenes results in more than ten times more yield than phenylacetylene (Boyd et al. (1991)). The biphenyl dioxygenases, promising catalysts for bioremediation of sites contaminated with polychlorinated biphenyl (PCB), are highly active toward biphenyl and mono-, di-, and tri-substituted PCB's, but showed no activity toward some more highly chlorinated PCB's (Mondello (1988)). The problem of low activity toward unnatural substrates is exacerbated by product inhibition, product toxicity, and low in vivo and in vitro enzyme stability (Jenkins et al. (1986); and Wahbi et al. (1996)).

Substrate Solubility

Most of the substrates of dioxygenase enzymes are insoluble in water. One solution to this problem is addition of ethanol or other co-solvent to the reaction mixture to solubilize the substrate (Woodley et al. (1996)). Another solution is use of an organic phase (e.g. dodecane) to partition the substrate and maintain a constant concentration in the aqueous phase (Woodley et al. (1992)). These methods can be deleterious to both the enzyme and the microbial host organism.

Stability

As is common with large proteins with quaternary structure, dioxygenases are particularly unstable. Toluene dioxygenase has a half life of about five hours at 30° C., and readily denatures at 50° C. When outside the cell, dioxygenase enzymes are rapidly destabilized by contact with air (Woodley et al. (1996)).

In Vitro vs. in vivo Bioconversion

Though in vitro bioconversions are often favored in the field of biocatalysis, several factors discourage the use of dioxygenase enzymes in vitro. Dioxygenase requires the expensive cofactor NADH for activity. In cells, this cofactor can be regenerated from a variety of carbon sources; however, for an in vitro system, additional enzymes must be provided for regeneration. Even when NADH is provided, the in vitro system for toluene conversion by toluene dioxygenase is five times less effective than the in vivo equivalent (Jenkins et al.(1986)). This may be a result of the effective dilution that occurs upon cell disruption, coupled with the low stability of dioxygenase outside the cell (Woodley et al. (1996)).

The problems of in vitro cofactor regeneration and substrate insolubility will be best solved through bioreactor design techniques. However, the biggest obstacle to implementation of dioxygenase enzymes is the enzyme itself, namely, its low activity toward industrially useful substrates, and its low stability at room temperature. Numerous studies have proven the usefulness of directed evolution in engineering these exact properties. However, the lack of rapid and sensitive screening methods to select evolved enzymes with the desired properties impairs the evolution of more industrially competent enzymes.

### The Gibbs Assay

An assay for the detection of phenol and hydroxypyridine derivatives was first reported by Gibbs in 1927. The assay utilizes 2,6-dichloro-p-benzoquinone (the “Gibbs reagent”), and has been extensively investigated by many researchers (Dacre (1971); Josephy (1984, No. 1); and Palagi et al. (1994, 1999)). The Gibbs reaction is sensitive and specific on both phenol and hydroxypyridine derivatives and results in indophenole derivatives as final products in a quantitative manner. It has been utilized for monitoring phenol derivatives in analysis of drug metabolism (Adam et al. (1981); and Belal (1984)), phenol exposure of workers (Orlewski et al. (1982); and Kobylianski et al. (1994)), enzymatic activities (Boyd et al. (19840; Velosy (1985); Kayser et al. (1993); Gilbert et al. (1998); and Singh et al. (1974)), and for other applications (Miller et al. (1990); and Wojtaszek et al. (1992)), as well as identification of chemical structure (Coop et al. (1995); Flavin et al. (1996); Uemura et al. (1988); Suzuki et al. (1992); Tadera et al. (1988); and Umezawa et al. (1987)), and detection of anti-oxidants (Josephy et al. (1984, No. 2), opium alkaloids (Baggi et al. (1976)), barbiturates (Vinson et al. (1977)) and catechols (Johnston et al. (1987)). Also, it has been found that the Gibbs reagent reacts specifically with sulfhydryl groups through a different mechanism (Harfoush (1986)), and this reaction was successfully used as a tool for detection of compounds having sulfhydryl groups (Hallit et al. (1981); McAllister (1951); and Searle (1955)). Dicumarol, a bioactive molecule, is known to be as a good catalyst for the degradation of the Gibbs reagent. The detection method for Dicumarol was developed by measurement of the degradation rate of Gibbs reagent (Panadero et al. (1993)). Although piperadine can be determined with this reagent, the mechanism is unknown (Baggi et al. (1974)).

Recently, Quintana et al. (1997) proposed that the Gibbs assay was applicable for the detection of phenols and catechols from biotransformation in a 96-well plate format. Using this reaction, Quintana et al. developed a screening method for microorganisms exhibiting dioxygenase activity toward aromatic compounds. In this system, however, the color formation is dependent on the small amount of phenol which arises from the corresponding benzene cis-dihydrodiols by slow spontaneous dehydration. Because only a small amount of phenol is produced, this method may not have the sensitivity required for screening libraries in which only low activity variants are expected (68).

A key requirement for finding improved oxidation biocatalysts in nature or by directed evolution is the availability of sensitive and rapid screening methods. The present invention, based on the surprising discovery of modifications of the Gibbs assay which increase the applicability and sensitivity of the method, addresses these and other problems in the art.

### SUMMARY OF THE INVENTION

The invention provides for a new screening method for oxidation enzymes, particularly useful for screening of new and improved oxidation enzymes. According to the methods of the invention, oxidation enzymes are screened or detected, or evaluated for their activity or other features, using a Gibbs assay with a novel and enhanced sensitivity. The invention further provides for high-throughput assays with a high sensitivity, and novel applications for the Gibbs assay. The resulting products are colored, and therefore easily detectable and quantifyable. Examples showing the principles for the methods of the invention are provided in FIGS. 14.

The invention provides for a method in which a product of a reaction catalyzed by an oxidation enzyme is converted into a modified product, preferably a phenol or a catechol (1,2-dihydroxybenzene), which is subsequently contacted with the Gibbs reagent (2,6-dichloroquinone-4-chloroimide). In a preferred embodiment of the invention, the product of the oxidation reaction is a cis-dihydrodiol. In other preferred embodiments, the product is a halogenated, aminated, carboxylated, or alkylated aromatic compound.

The invention also provides for both liquid and solid phase assays, in which an oxidized product of a reaction catalyzed by an oxidation enzyme is detected by contacting the product with Gibbs reagent. The liquid phase assay preferably comprises a step in which the product is modified to be more easily detectable by the Gibbs reagent. This is preferably accomplished by acidification to convert the oxidized product into a phenol, or the addition of an enzyme which catalyzes the conversion of the oxidized product into a phenol or a catechol, or acidification combined with the addition of an enzyme, prior to providing Gibbs reagent. For example, if the oxidized product is a cis-dihydridiol or anthranilic acid, respective enzymes which catalyze the conversion into a catechol may be cis-dihydrodiol dehydrogenase and anthranilate monooxygenase. If the oxidized product is halogenated benzene, a dehalogenase may be used to convert the product into a phenol. In addition, benzoic acid or alkylated benzene products may be converted into phenols by use of cytochrome P450 and peroxidases.

The solid-phase assay may comprise transforming cells with a plasmid encoding for an oxidation enzyme such as, e.g., a monooxygenase or dioxygenase enzyme, and growing the transformed cells on an agar plate. The cells on the agar plate can thereafter be transferred to a membrane, and the membrane contacted with a substrate which is oxidized by the oxidation enzyme. If the resulting oxidized product is a phenol, the product may be directly detected using Gibbs reagent. However, in a preferred embodiment, the solid phase assay comprises a step in which the oxidized product is modified to be more easily detectable by the Gibbs reagent. For example, if the resulting product is a cis-dihydridiol, an acidification step or a step in which cis-dihydrodiol dehydrogenase is provided, or a step comprising both acidification and the addition of an enzyme, can be added to enhance the sensitivity of the subsequent detection with Gibbs reagent. In a preferred embodiment, cis-dihydrodiol dehydrogenase is provided by co-transforming the cells with the gene encoding for cis-dihydrodiol dehydrogenase in the transformation step. This can be accomplished by transforming the cells with a plasmid which comprises genes for both the oxidation enzyme and cis-dihydrodiol dehydrogenase. In other embodiments where the product is a halogenated or alkylated benzene, benzoic acid, or anthranilic acid, the host cells may be co-transformed with plasmids encoding for suitable enzymes selected from, e.g., dehalogenase, anthranilate monooxygenase, cytochrome P450, or peroxidase, to convert the product into a phenol or a catechol.

The invention further provides for a novel method for screening of oxidation enzymes which catalyze oxidation of phenyl ethers. The product of a hydroxylation reaction which occurs on the aromatic part of the substrate is directly detectable by contacting with Gibbs reagent. If the oxidation takes place at the ether part, the resulting compound may dissociate spontaneously into a phenol and an aldehyde, of which the phenol can be detected by a Gibbs assay. General aspects of this embodiment of the invention are shown in FIG. 12.

The invention also provides for the detection for screening for thioesterases, which may catalyze the conversion of thioesters into carboxylic acid-containing compounds, wherein the leaving thiol group is detected by Gibbs reagent.

The invention is particularly well suited for screening a large number of naturally occurring or mutated oxidation enzymes to determine relative enzyme activities with respect to a substrate, and in particular to establish which enzymes exhibit the highest activity with respect to a given substrate. The invention is applicable to both monooxygenases or dioxygenases and can be used to detect oxygenated compounds formed by hydroxylation, epoxidation, and sulfoxidation. Hydroxylation enzymes are one preferred species of enzymes for the invention.

The above features and many other attendant advantages of the invention will become better understood by reference to the following detailed description when taken in conjunction with the accompanying drawings.

### BRIEF DESCRIPTION OF THE DRAWINGS

FIG. 1. Aromatic degradation by a dioxygenase pathway.

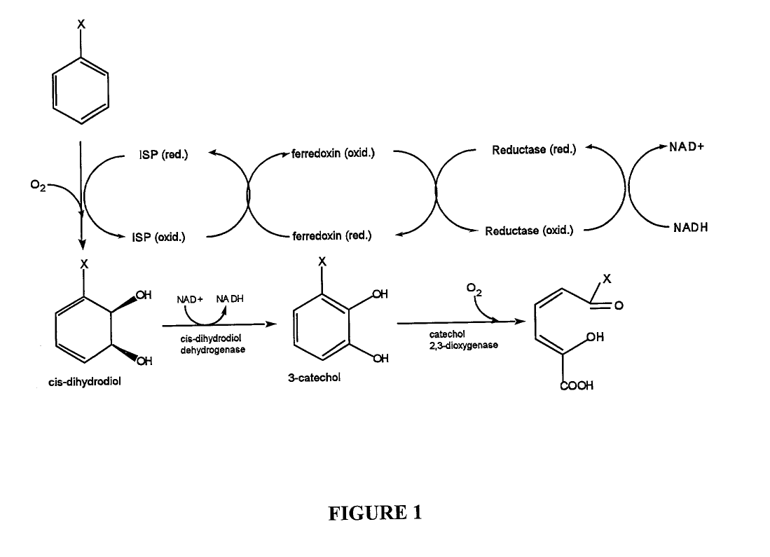

FIG. 2. Mechanism of a dioxygenase reaction.

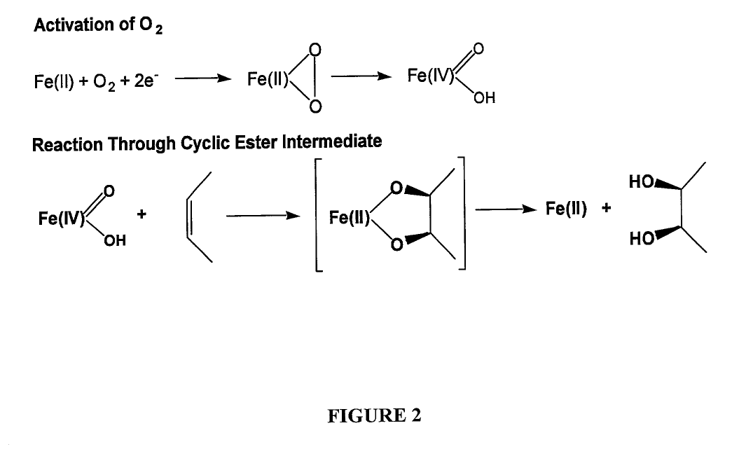

FIG. 3. Gibbs assay with acidification process according to the invention.

FIG. 4. Correlation between the concentration of toluene cis-dihydrodiol and absorbance at 590 nm of the product from the Gibbs assay. Open circle, with acidification; closed circle, without acidification.

FIG. 5. Variation in the activity of wild type toluene dioxygenase in a 96-well plate assay, measured by absorbance at 590 nm.

FIG. 6. Profile of the relative activity of toluene dioxygenase variants toward toluene. The activity of wild type enzyme was normalized to 100.

FIG. 7. Profile of the relative activity of toluene dioxygenase variants toward 4-picoline. The activity of the wild-type enzyme was normalized to 100.

FIG. 8. Results from solid-phase Gibbs assay. A. Colonies expressing mutant pXTD14 were assayed for activity toward chlorobenzene using method I. Arisawa (1999) applied random mutagenesis to the gene coding for the α subunit of toluene dioxygenase (todC1). B. Colonies expressing wild-type pJMJ2 were assayed for activity toward chlorobenzene using Method I. C. Colonies expressing wild-type pXTD12 were assayed for activity toward toluene using Method II. Inset represents millimeter scale. Photos were taken using an AGFA digital camera.

FIG. 9. Elimination of large-scale contrast across a raw image acquired using a Fluor-S™ Multiimager. The image used was a screening result for wild-type colonies exposed to chlorobenzene vapor.

FIG. 10. Scheme of the P450 catalyzed hydroxylation of hydrocarbons.

FIG. 11. Proposed scheme for high-throughput enantioselectivity measurement.

FIG. 12. Scheme of the P450 catalyzed hydroxylation of phenolic compounds and subsequent detection with Gibbs reagent.

FIG. 13. Expression plasmids used in the Examples.

FIG. 14. Summary depiction of some preferred embodiments of the invention. See also FIG. 3. A. Bioconversion catalyzed by dioxygenase enzymes, shown generically for a monocyclic, monosubstituted compound. B. Acidification method for quantitation of dihydrodiol. At low pH, dihydrodiol undergoes dehydration to either an o- or m-phenol, which is detectable with Gibbs reagent. C. Dehydrogenase method for quantitation of cis-dihydrodiol. Cis-dihydrodiol dehydrogenase converts cis-dihydrodiol to a catechol, which reacts with Gibbs reagent.

FIG. 15. A. UV spectrum of colored products resulting from liquid-phase screening for toluene dioxygenase activity toward chlorobenzene. B. Plot showing linear correlation between chlorobenzene cis-dihydrodiol concentration and its absorbance at 652 mn after acidification and coupling with Gibbs reagent in minimal medium.

FIG. 16. A. Unmodified digital image showing a screening result for colonies expressing wil-type pJMJ2 (toluene dioxygenase). B. Enlargement of squared region in A. C. Digital image after processing, as described in Example 3. D. Enlargement of squared region in C. The Petri dish was 15 cm in diameter.

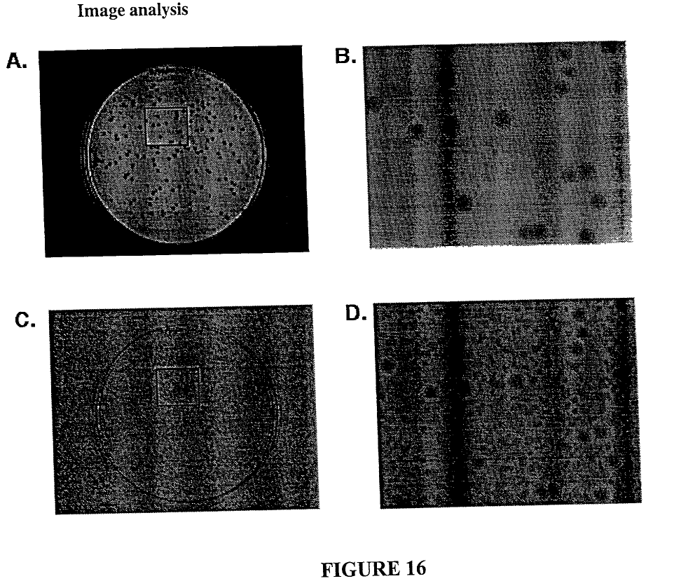

FIG. 17. A. Histogram represenation of wild-type activity measurements from solid- and liquid-phase methods. Ninety-six colonies expressingwild-type toluene dioxygenase were screened using both methods. The standard deviation of activity measurements was 9.0% with the liquid-phase method and 5.3% with the solid-phase method. B. Comparison of mutant activity measurements generated by both the solid and liquid-phase methods. Mutants were created by error-prone PCR applied to the gene encoding for the large subunit of toluene dioxygenase. One-hundred sixty mutants were screened with the liquid-phase method, and 1899 were screened with the solid-phase method.

### DETAILED DESCRIPTION OF THE INVENTION

The invention concerns methods which enable the identification of new naturally occurring oxidation or aromatic or cyclic-hydroxylating enzymes as well as novel and/or improved mutant enzymes obtained by directed evolution. In particular, the invention is well suited for evaluating the activity of enzymes that are capable of oxidizing aromatic substrates. The method utilizes the Gibbs reagent to identify samples containing the enzymes and, if desired, can be used for quantifying the relative activity of the samples in order to find improved catalysts. One of the novel features of the invention is the conversion of oxidized products such as cis-dihydrodiols, which cannot be efficiently detected by the Gibbs reagent, into phenols and/or catechols which are efficiently detected by the Gibbs assay. The screening method can be implemented in either the solid phase (e.g., on culture plates or membranes) or in solution (e.g., in 384-well microtiter plates). The solid phase screen is expected to be particularly useful for rapid screening of enzyme libraries, for example produced by random mutagenesis or recombination (DNA shuffling). It could also advantageously be used for screening libraries for the presence of hydroxylating enzymes. The application of the screening methods of the invention on the directed evolution of a dioxygenase enzyme to improve the catalytic activity towards a nonnatural substrate is presented herein.

### Definitions

Generally, a chemical compound used in or obtained by the methods of the invention includes derivatives of the compound, e.g., wherein the compound contains one or more substituents not specifically identified, or larger structures in which the compound comprises one part. Thus, “a phenol” means any compound which contains a phenol group, including phenol, phenol derivatives, and compounds derivatized with a phenol group. Likewise, “a catechol” means any compound which contains a catechol group, including catechol, catechol derivatives, and compounds derivatized with a catechol group. “A dihydrodiol” means any compound which contains a dihydrodiol group, including aromatic, aliphatic, cyclic hydrocarbons, and heterocyclic compounds, which contain a dihydrodiol feature. A preferred dihydrodiol is cis-dihydrodiol. An “aromatic hydrocarbon” includes substituted aromatic hydrocarbons, which may also be bicyclic in which at least one cyclic moiety is aromatic. A “halogenated ethylene” means any compound which contains a halogenated ethylene group. Similarly, the term “phenyl ether” includes phenyl ether, substituted phenyl ethers, and compounds derivatized with a phenyl ether.

“Acidic” conditions means conditions under which the pH of a particular environment, e.g., an aqueous or non-aqueous solution, is lower than pH 7. Acidic conditions are used according to the methods of the invention to promote the conversion of a dihydrodiol into a phenol. For this purpose, a pH lower than 7, preferably lower than 4, and even more preferably lower than 3, is suitable. For a cis-dihydrodiol, a pH lower than 4, preferably about 2.5, is preferred. Suitable means to render experimental conditions acidic include, but are not limited to, the addition of HCl, H₂SO₄, or other commonly used acids, in aqueous solution or in gaseous phase.

A “modified product” herein means a product which has been modified to remove one or more atoms from, or to add one or more atoms to, the product. For example, a cis-dihydrodiol product may be modified by removing 2 hydrogens and one oxygen atoms, i.e., a water molecule. Generally, a product from an oxidation reaction is modified to enable or enhance detection of the product being formed via a Gibbs assay.

As used herein, “about” or “approximately” shall mean within 50 percent, preferably within 20 percent, more preferably within 5 percent, and even more preferably within 5 percent of a given value or range.

Molecular Biology Definitions

The general molecular biology and genetic engineering tools and techniques discussed here, including transformation and expression, the use of host cells, vectors, expression systems, etc., are well known in the art.

A “protein” or “polypeptide”, which terms are used interchangeably herein, comprises one or more chains of chemical building blocks called amino acids that are linked together by chemical bonds called peptide bonds.

An “enzyme” means any substance, preferably composed wholly or largely of protein, that catalyzes or promotes, more or less specifically, one or more chemical or biochemical reactions. The term “enzyme” can also refer to a catalytic polynucleotide (e.g. RNA or DNA). A “test” enzyme is a substance that is tested to determine whether it has properties of an enzyme.

Proteins and enzymes can be made in a host cell using instructions in DNA and RNA, according to the genetic code. “Transcription” is the process by which a DNA sequence or gene having instructions for a particular protein or enzyme is “transcribed” into a corresponding sequence of RNA. “Translation” is the process by which the RNA sequence is “translated” into the sequence of amino acids which form the protein or enzyme.

A “native” or “wild-type” protein, enzyme, polynucleotide, gene, or cell, means a protein, enzyme, polynucleotide, gene, or cell that occurs in nature.

A “parent” protein, enzyme, polynucleotide, gene, or cell, is any protein, enzyme, polynucleotide, gene, or cell, from which any other protein, enzyme, polynucleotide, gene, or cell, is derived or made, using any methods, tools or techniques, and whether or not the parent is itself native or mutant. A parent polynucleotide or gene can encode for a parent protein or enzyme.

A “mutant”, “variant” or “modified” protein, enzyme, polynucleotide, gene, or cell, means a protein, enzyme, polynucleotide, gene, or cell, that has been altered or derived, or is in some way different or changed, from a parent protein, enzyme, polynucleotide, gene, or cell. A mutant protein or enzyme is usually, although not necessarily, expressed from a mutant polynucleotide or gene.

A “mutation” means any process or mechanism resulting in a mutant protein, enzyme, polynucleotide, gene, or cell. This includes any mutation in which a protein, enzyme, polynucleotide, or gene sequence is altered, any protein, enzyme, polynucleotide, or gene sequence arising from a mutation, any expression product (e.g. protein or enzyme) expressed from a mutated polynucleotide gene sequence, and any detectable change in a cell arising from such a mutation.

Regarding genetic material, “mutant” and “mutation” includes polynucleotide alterations arising within a protein-encoding region of a gene as well as alterations in regions outside of a protein-encoding sequence, such as, but not limited to, regulatory sequences. “Mutant” also includes a “silent” mutant and “sequence-conservative variants”, which is a mutant polynucleotide sequence that, upon translation, is not reflected in an altered amino acid sequence. Such silent mutations can occur when one amino acid corresponds to more than one codon.

“Function-conservative variants” are proteins or enzymes in which a given amino acid residue has been changed without altering overall conformation and function of the protein or enzyme, including, but not limited to, replacement of an amino acid with one having similar properties (such as, for example, acidic, basic, hydrophobic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Amino acids other than those indicated as conserved may differ in a protein or enzyme so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide or enzyme which has at least 60% amino acid identity as determined by BLAST or FASTA algorithms, preferably at least 75%, most preferably at least 85%, and even more preferably at least 90%, and which has the same or substantially similar properties or functions as the native or parent protein or enzyme to which it is compared.

A “property” or “feature” of a protein or enzyme, wild-type or mutated, means a property or feature, preferably detectable in a screening test, associated with the protein. Protein properties and features include, but are not limited to, the ability of the protein to fold correctly, the stability of the protein in a certain media and/or over time, the expression level or yield of a protein expressed by a host cell, functionality (i.e., whether the protein is functional or non-functional), and, in the case of a enzyme, enzyme activity and substrate specificity.

The “activity” of an enzyme is a measure of its ability to catalyze a reaction, and may be expressed as the rate at which the product of the reaction is produced. For example, enzyme activity can be represented as the amount of product produced per unit of time, per unit (e.g. concentration or weight) of enzyme.

The “stability” of an enzyme means its ability to function, over time, in a particular environment or under particular conditions. One way to evaluate stability is to assess its ability to resist a loss of activity over time, under given conditions. Enzyme stability can also be evaluated in other ways, for example, by determining the relative degree to which the enzyme is in a folded or unfolded state. Thus, one enzyme is more stable than another, or has improved stability, when it is more resistant than the other enzyme to a loss of activity under the same conditions, is more resistant to unfolding, or is more durable by any suitable measure. For example, a more “thermally stable” or “thermostable” enzyme is one that is more resistant to loss of structure (unfolding) or function (enzyme activity) when exposed to heat or an elevated temperature. One way to evaluate this is to determine the “melting temperature” or Tₘ for the protein. The melting temperature, also called a midpoint, is the temperature at which half of the protein is unfolded from its fully folded state. This midpoint is typically determined by calculating the midpoint of a titration curve that plots protein unfolding as a function of temperature. Thus, a protein with a higher Tₘ requires more heat to cause unfolding and is more stable or more thermostable. Stated another way, a protein with a higher Tₘ indicates that fewer molecules of that protein are unfolded at the same temperature as a protein with a lower Tₘ, again meaning that the protein which is more resistant to unfolding is more stable (it has less unfolding at the same temperature). Another measure of stability is T_{½}, which is the transition midpoint of the inactivation curve of the protein as a function of temperature. T_{½} is the temperature at which the protein loses half of its activity. Thus, a protein with a higher T_{½} requires more heat to deactivate it, and is more stable or more thermostable. Stated another way, a protein with a higher T_{½} indicates that fewer molecules of that protein are inactive at the same temperature as a protein with a lower T_{½}, again meaning that the protein which is more resistant to deactivation is more stable (it has more activity at the same temperature). These assays are also called “thermal shift” assays, because the inactivation or unfolding curve, plotted against temperature, is “shifted” to higher or lower temperatures when stability increases or decreases. Thermostability can also be measured in other ways. For example, a longer half-life (t_{½}) for the enzyme's activity at elevated temperature is an indication of thermostability.

A “functional” protein or enzyme is capable of displaying biological activity, such as, for example, participating in a designated biochemical reaction. Generally, a screening test can be used to detect and/or evaluate whether a protein is functional or not.

The term “substrate” means any substance or compound that is converted or meant to be converted into another compound by the action of an enzyme catalyst. The term includes aromatic and aliphatic compounds, and includes not only a single compound, but also combinations of compounds, such as solutions, mixtures and other materials which contain at least one substrate. Exemplary and non-limiting aromatic substrates of the invention include halogenated benzenes (fluoro-, chloro-, bromo-, and iodo-benzene), benzamide, benzoic acid, anthranilic acid, 2-naphtoic acid, pyridine, biphenyl, heterocyclic compounds such as 4-picoline, and various mono- and di-substituted monocyclic aromatics such as toluene, benzene, t-butyl benzene, 1,2,4-trimethylbenzene, and p-methoxybenzoic acid. Preferred substrates include benzene, chlorobenzene, toluene, and 4-picoline.

The term “cofactor” means any non-protein substance that is necessary or beneficial to the activity of an enzyme. A “coenzyme” means a cofactor that interacts directly with and serves to promote a reaction catalyzed by an enzyme. Many coenzymes serve as carriers. For example, NAD⁺, NADP⁺, and FAD, corresponding to NADH, NADPH, and FADH₂ in their respective reduced forms, carry hydrogen atoms from one enzyme to another. An “ancillary protein” means any protein substance that is necessary or beneficial to the activity of an enzyme.

An “oxidation reaction” or “oxygenation reaction”, as used herein, is a chemical or biochemical reaction involving the addition of oxygen to a substrate, to form an oxygenated or oxidized substrate or product. An oxidation reaction is typically accompanied by a reduction reaction (hence the term “redox” reaction, for oxidation and reduction). A compound is “oxidized” when it receives oxygen or loses electrons. A compound is “reduced” when it loses oxygen or gains electrons. According to the invention, oxidation reactions are preferably oxygenation reactions which add oxygen to a substrate. Oxygen typically donates electrons in ionic form as OH⁻ or O₂²⁻. Conceptually, electrons (negatively charged subatomic particles) may also be lost or gained via the transfer of protons (positively charged subatomic particles), for example as hydrogen ions (H⁺ of H₂²⁺). An “ion” is an atom or molecule with a net positive or negative charge, i.e. it has excess electrons (a negative charge) or is missing electrons (a positive charge). Thus, an oxidation reaction can also be called an “electron transfer reaction” and encompass the loss or gain of electrons (e.g. oxygen) or protons (e.g. hydrogen) from a substance. Preferred oxidized compounds of the invention are those which are “oxygenated”, meaning they have received oxygen.

The terms “oxygen donor”, “oxidizing agent” and “oxidant” mean a substance, molecule or compound which donates oxygen to a substrate in an oxidation reaction. Typically, the oxygen donor is reduced (accepts electrons). Exemplary oxygen donors, which are not limiting, include molecular oxygen or dioxygen (O₂) and peroxides, including alkyl peroxides such as t-butyl peroxide, and hydrogen peroxide (H₂O₂). A peroxide is any compound having two oxygen atoms bound to each other.

An “oxidation enzyme” is an enzyme that catalyzes one or more oxidation reactions, typically by adding, inserting, contributing or transferring oxygen from a source or donor to a substrate, or by reducing the relative amount of hydrogen in the substrate, or by increasing the proportion of the electronegative constituent in a compound. Oxidation reactions include, but are not limited to, aromatic oxidative dehalogenation and aromatic oxidative dealkylation. Enzymes which catalyze oxidation reactions include oxidoreductases or redox enzymes, and encompasses oxygenases, dehydrogenases or reductases, oxidases and peroxidases. An “oxygenase” is an oxidation enzyme that catalyzes the addition of oxygen to a substrate compound. A “dioxygenase”, is an oxygenase enzyme that adds two atoms of oxygen to a substrate. A “monooxygenase” adds one atom of oxygen to a substrate. An “oxidase” is an oxidation enzyme that catalyzes a reaction in which molecular oxygen (dioxygen or O₂) is reduced, for example by donating electrons to (or receiving protons from) hydrogen.

Preferred oxidation enzymes for screening using the methods of the invention include, without limitation, oxygenases (dioxygenases and monooxygenases), including hydroxylases, epoxidases, and sulfoxidases, which catalyze, respectively, hydroxylation, epoxidation, and sulfoxidation reactions. Of these, monooxygenases, hydroxylases, and dioxygenases are preferred. Exemplary oxidation enzymes include, without limitation, native or modified chloroperoxidase (CPO), methane monooxygenases (MMOs), toluene monooxygenase (e.g., toluene o-monooxygenase), toluene dioxygenases (TDO), naphthalene dioxygenases (NDO), and biphenyl dioxygenases; phenol hydroxylase, dehalogenase, and microperoxidase (dehalogenation); and cytochrome P450 and microperoxidase (dealkylation). A preferred oxidation enzyme is native or modified toluene dioxygenase.

Moreover, dehydrogenases may be used according to the methods of the invention to promote the conversion of compounds, including, but not limited to, dihydrodiols such as cis-dihydrodiol, to catechols. A preferred dehydrogenase is cis-dihydrodiol dehydrogenase. Other enzymes which promote the conversion of an oxidation product into a phenol or a catechol may also be used, including dehalogenases such as tryptophan dehalogenase, anthranilate monooxygenase, cytochrome P450, or peroxidases.

A “detection agent” means any substance which produces a detectable signal. Such a signal could be, for example, electromagnetic radiation, or a change in electromagnetic radiation, most notably visible light, by any mechanism, including color change, UV absorbance, fluorescence and phosphorescence. Preferably, a luminescent substance according to the invention produces a detectable color, fluorescence or UV absorbance. A preferred detection agent is the Gibbs reagent (2,6-dichloroquinone-4-chloroimide) (Gibbs (1927)).

“DNA” (deoxyribonucleic acid) means any chain or sequence of the chemical building blocks adenine (A), guanine (G), cytosine (C) and thymine (T), called nucleotide bases, that are linked together on a deoxyribose sugar backbone. DNA can have one strand of nucleotide bases, or two complimentary strands which may form a double helix structure. “RNA” (ribonucleic acid) means any chain or sequence of the chemical building blocks adenine (A), guanine (G), cytosine (C) and uracil (U), called nucleotide bases, that are linked together on a ribose sugar backbone. RNA typically has one strand of nucleotide bases.

A “polynucleotide” or “nucleotide sequence” is a series of nucleotide bases (also called “nucleotides”) in DNA and RNA, and means any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotide, and both sense and anti-sense polynucleotide (although only sense stands are being represented herein). This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases, for example thio-uracil, thio-guanine and fluoro-uracil.

The polynucleotides herein may be flanked by natural regulatory sequences, or may be associated with heterologous sequences, including promoters, enhancers, response elements, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

A “codon” is a triplet of nucleotides corresponding to an amino acid. Each amino acid is represented in DNA or RNA by one or more codons. The genetic code has some redundancy, also called degeneracy, meaning that most amino acids have more than one corresponding codon. For example, the amino acid lysine (Lys) can be coded by the nucleotide triplet or codon AAA or by the codon AAG.

The “reading frame” describes the way that a nucleotide sequence is grouped into codons. Because the nucleotides in DNA and RNA sequences are read in groups of three for protein production, it is important to begin reading the sequence at the correct amino acid, so that the correct triplets are read.

A “coding sequence” or a sequence “encoding” a polypeptide, protein or enzyme is a nucleotide sequence that, when expressed, results in the production of that polypeptide, protein or enzyme, i.e., the nucleotide sequence encodes an amino acid sequence for that polypeptide, protein or enzyme. A coding sequence is “under the control” of transcriptional and translational control sequences in a cell when RNA polymerase transcribes the coding sequence into mRNA, which is then trans-RNA spliced and translated into the protein encoded by the coding sequence. Preferably, the coding sequence is a double-stranded DNA sequence which is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence will usually be located 3′ to the coding sequence.

The term “gene”, also called a “structural gene” means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription. A gene encoding a protein of the invention for use in an expression system, whether genomic DNA or cDNA, can be isolated from any source, particularly from a human cDNA or genomic library. Methods for obtaining genes are well known in the art, e.g., Sambrook et al. (52).

Any animal cell potentially can serve as the nucleic acid source for the molecular cloning of the gene of interest. The DNA may be obtained by standard procedures known in the art, such as from cloned DNA (e.g., a DNA “library”), from cDNA library prepared from tissues with high level expression of the protein, by chemical synthesis, by cDNA cloning, or by the cloning of genomic DNA, or fragments thereof, purified from the desired cell. Clones derived from genomic DNA may contain regulatory and intron DNA regions in addition to coding regions; clones derived from cDNA will not contain intron sequences.

In the molecular cloning of the gene from genomic DNA, DNA fragments are generated, some of which will encode the desired gene. The DNA may be cleaved at specific sites using various restriction enzymes. Alternatively, one may use DNAse in the presence of manganese to fragment the DNA, or the DNA can be physically sheared, as for example, by sonication. The linear DNA fragments can then be separated according to size by standard techniques, including but not limited to, agarose and polyacrylamide gel electrophoresis and column chromatography.

A transcriptional or translational “control sequence” is a DNA regulatory sequence, such as a promoter, enhancer, terminator, and the like, that provide for the expression of a coding sequence in a host cell. In eukaryotic cells, polyadenylation signals are control sequences.

A “promoter sequence” is a DNA regulatory region capable of binding RNA polymerase in a cell and initiating transcription of a downstream (3′ direction) coding sequence. For purposes of defining this invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase. As described above, promoter DNA is a DNA sequence which initiates, regulates, or otherwise mediates or controls the expression of the coding DNA. A promoter may be “inducible”, meaning that it is influenced by the presence or amount of another compound (an “inducer”). For example, an inducible promoter includes those which initiate or increase the expression of a downstream coding sequence in the presence of a particular inducer compound. A “leaky” inducible promoter is a promoter that provides a high expression level in the presence of an inducer compound and a comparatively very low expression level, and at minimum a detectable expression level, in the absence of the inducer.

Polynucleotides are “hybridizable” to each other when at least one strand of one polynucleotide can anneal to another polynucleotide under defined stringency conditions. Stringency of hybridization is determined, e.g., by a) the temperature at which hybridization and/or washing is performed, and b) the ionic strength and polarity (e.g., formamide) of the hybridization and washing solutions, as well as other parameters. Hybridization requires that the two polynucleotides contain substantially complementary sequences; depending on the stringency of hybridization, however, mismatches may be tolerated. Typically, hybridization of two sequences at high stringency (such as, for example, in an aqueous solution of 0.5×SSC at 65° C.) requires that the sequences exhibit some high degree of complementarity over their entire sequence. Conditions of intermediate stringency (such as, for example, an aqueous solution of 2×SSC at 65° C.) and low stringency (such as, for example, an aqueous solution of 2×SSC at 55° C.), require correspondingly less overall complementarity between the hybridizing sequences. (1×SSC is 0.15 M NaCl, 0.015 M Na citrate.) Polynucleotides that “hybridize” to the polynucleotides herein may be of any length. In one embodiment, such polynucleotides are at least 10, preferably at least 15 and most preferably at least 20 nucleotides long. In another embodiment, polynucleotides that hybridizes are of about the same length. In another embodiment, polynucleotides that hybridize include those which anneal under suitable stringency conditions and which encode polypeptides or enzymes having the same function, such as the ability to catalyze an oxidation, oxygenase, or coupling reaction of the invention.

The term “DNA reassembly” is used when recombination occurs between identical sequences. “DNA shuffling” refers herein to a group of in vitro and in vivo methods involving recombination of nucleic acid species. Such methods can be employed to generate polynucleotide molecules having variant sequences of the invention.

The term “host cell” means any cell of any organism that is selected, modified, transformed, grown, or used or manipulated in any way, for the production of a substance by the cell, for example the expression by the cell of a gene, a DNA or RNA sequence, a protein or an enzyme.

The term “expression system” means a host cell and compatible vector under suitable conditions, e.g. for the expression of a protein coded for by foreign DNA carried by the vector and introduced to the host cell. Common expression systems include bacteria (e.g. E. coli and B. subtilis) or yeast (e.g. S. cerevisiae) host cells and plasmid vectors, and insect host cells and Baculovirus vectors. As used herein, a “facile expression system” means any expression system that is foreign or heterologous to a selected polynucleotide or polypeptide, and which employs host cells that can be grown or maintained more advantageously than cells that are native or heterologous to the selected polynucleotide or polypeptide, or which can produce the polypeptide more efficiently or in higher yield. For example, the use of robust prokaryotic cells to express a protein of eukaryotic origin would be a facile expression system. Preferred facile expression systems include E. coli, B. subtilis and S. cerevisiae host cells and any suitable vector.

The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences, such as start, stop, promoter, signal, secretion, or other sequences used by a cell's genetic machinery. The gene or sequence may include nonfunctional sequences or sequences with no known function. A host cell that receives and expresses introduced DNA or RNA has been “transformed” and is a “transformant” or a “clone.” The DNA or RNA introduced to a host cell can come from any source, including cells of the same genus or species as the host cell, or cells of a different genus or species.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.”

A common type of vector is a “plasmid”, which generally is a self-contained molecule of double-stranded DNA, that can readily accept additional (foreign) DNA and which can readily introduced into a suitable host cell. A plasmid vector often contains coding DNA and promoter DNA and has one or more restriction sites suitable for inserting foreign DNA. Promoter DNA and coding DNA may be from the same gene or from different genes, and may be from the same or different organisms. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wisc.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes. Preferred vectors are described in the Examples, and include without limitations pXTD8, pXTD12, pXTD14, pJMJ2, and pJMJ8. Other vectors may be employed as desired by one skilled in the art. Routine experimentation in biotechnology can be used to determine which vectors are best suited for used with the invention, if different than as described in the Examples. In general, the choice of vector depends on the size of the polynucleotide sequence and the host cell to be employed in the methods of this invention.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed” by the cell. A polynucleotide or polypeptide is expressed recombinantly, for example, when it is expressed or produced in a foreign host cell under the control of a foreign or native promoter, or in a native host cell under the control of a foreign promoter.

“Isolation” or “purification” of a polypeptide or enzyme refers to the derivation of the polypeptide by removing it from its original environment (for example, from its natural environment if it is naturally occurring, or form the host cell if it is produced by recombinant DNA methods). Methods for polypeptide purification are well-known in the art, including, without limitation, preparative disc-gel electrophoresis, isoelectric focusing, HPLC, reversed-phase HPLC, gel filtration, ion exchange and partition chromatography, and countercurrent distribution. For some purposes, it is preferable to produce the polypeptide in a recombinant system in which the protein contains an additional sequence tag that facilitates purification, such as, but not limited to, a polyhistidine sequence. The polypeptide can then be purified from a crude lysate of the host cell by chromatography on an appropriate solid-phase matrix. Alternatively, antibodies produced against the protein or against peptides derived therefrom can be used as purification reagents. Other purification methods are possible. A purified polynucleotide or polypeptide may contain less than about 50%, preferably less than about 75%, and most preferably less than about 90%, of the cellular components with which it was originally associated. A “substantially pure” enzyme indicates the highest degree of purity which can be achieved using conventional purification techniques known in the art.

### Screening Methods

The assays or screening methods of the invention are applicable to a variety of enzymes, and is especially well suited for screening oxidation enzymes such as monooxygenases and dioxygenases which are capable of oxidizing a substrate, e.g., by hydroxylation.

High-throughput Screening Methods

To either discover novel enzymes from nature or improve known enzymes through directed evolution, high-throughput screening methods are required. Key aspects of screening technology include: 1) throughput, 2) accuracy and reproducibility of the measurements, 3) extendability to new problems. HPLC can be used to determine the presence of enzymatic degradation products, but its use in screening libraries is limited by the high cost of carrier liquid and relatively low throughput. Also, it can be difficult to reoptimize the system upon taking on a new problem. The screening methodologies provided herein can be used for making tens of thousands of highly accurate measurements per day. The chemistry employed is highly versatile, and may find application in screening for various classes of enzymatic reactions, such as dihydroxylation, monohydroxylation, dealkylation, and oxidative dehalogenation. The methods can also be useful for the survey of the aromatic compounds present in an environmental sample, and for monitoring the production of mono- or di-oxygenated aromatic or heterocyclic compounds.

The high-throughput screening methods described in this report can be used for screening libraries of mono- or dioxygenase enzymes for increased activity toward certain aromatic substrates as well as other suitable substrates. The invention provides for a simple and reliable colorimetric assay of mono- or di-oxygenated aromatic compounds produced by enzymatic reactions in bacterial biotransformations. This assay system involves color formation caused by the condensation of these compounds with 2,6-dichloroquinone-4-chloroimine (Gibbs reagent), and hence, it is superior to the HPLC system in terms of simplicity, flexibility and cost. The products of this modified Gibbs reaction are colored and are therefore easily detectable by a variety of means, including spectrometry, visual inspection, and digital imaging. Also, this method has a detection limit of 10 μM or less of cis-dihydrodiol, i.e., a concentration of 10 μM of cis-dihydrodiol is readily detectable after bioconversion. Thus, the methods of the invention are quantitative, and more sensitive than methods based on HPLC analysis.

General Principles of the Screening Methods of the Invention

In a broad aspect, the screening methods of the invention comprise combining, in any order, substrate, oxygen donor, test oxidation enzyme, and a method step to promote the conversion of the product of the oxidation reaction into a compound which can react with the Gibbs reagent. The inclusion of a step which promotes the conversion of the oxidation product into a compound which reacts with the Gibbs reagent provides a way to enable, or increase the sensitivity and efficacy of, the measurement of successful oxidation, that is, the formation of an oxygenated product from the substrate. The assay components can be placed in or on any suitable medium, carrier or support, and are combined under predetermined conditions. The conditions are chosen to facilitate, suit, promote, investigate or test the oxidation of the substrate by the oxygen donor in the presence of the test enzyme, and may be modified during the assay, for example to change conditions such as pH. In some embodiments, one or more cofactors, coenzymes and additional or ancillary proteins may be used to promote or enhance activity of the oxidation enzyme, the coupling enzyme, or both.

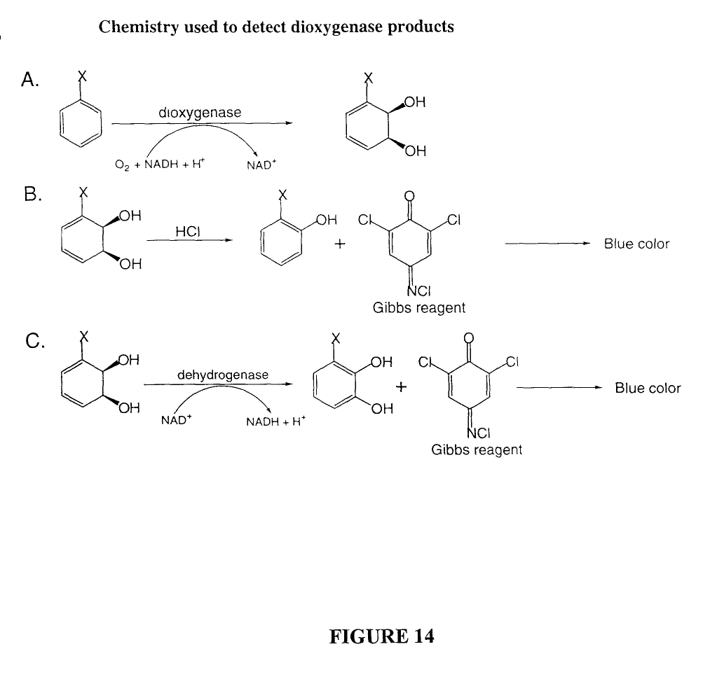

One of the key steps in the methods of the invention is the conversion of an oxidation product into a compound readily detected by the Gibbs reagent. Compounds which are readily detected by the Gibbs reagent include, but are not limited to, phenol and catechol, as well as halogenated or otherwise substituted phenol and catechol. The oxidation product may be converted into any of the foregoing Gibbs substrates by any means known in the art. For example, the oxidation product may be exposed to acidic conditions to remove one or more hydroxyl groups or other substituents from an aromatic ring. If the oxidation product is a dihydrodiol such as a cis-dihydrodiol (or “vic-dihydrodiol” as in “vicinal”), acidification would promote the conversion into a phenol. Alternatively, an enzyme may be used to remove one or more substitutents from a compound such as, but not limited to, an aromatic ring, or indeed to add one or more hydroxyl groups to a compound such as an aromatic substance, so that the oxidation product is converted into a phenol or a catechol. One example of the former strategy is the use of an oxidation enzyme to remove one or more hydrogen-containing substituents such as hydroxyl groups from, e.g., a benzene derivative. For instance, in a preferred embodiment, cis-dihydrodiol dehydrogenase is used to promote the formation of catechol from a cis-dihydrodiol, as outlined in FIG. 14.

Similarly, a halogenated benzene structure may be converted into a phenol by use of the corresponding dehalogenase (i.e., a chlorobenzene may be converted into a phenol using a dehalogenase specific for chlorinated compounds), to enable the detection of a compound with a Gibbs assay.

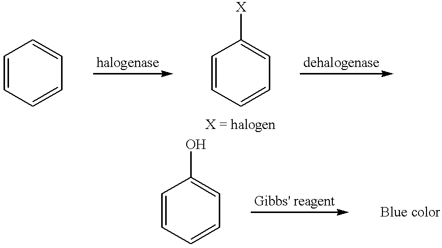

Also, anthranilate monooxygenase may be used to convert anthranilic acid into a catechol, which is detected by Gibbs reagent.

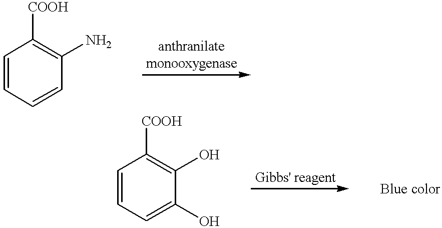

In addition, an aromatic structure such as, e.g., benzene, which is substituted with an alkyl or acid group may be converted into a phenol using cytochrome P450 or a peroxidase.

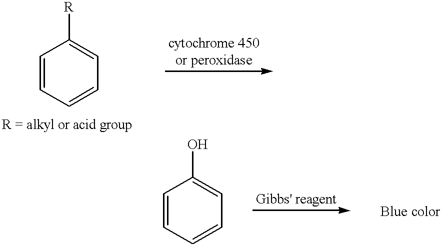

In an alternative embodiment, both an acidification and an enzyme step is performed, simultaneously or sequentially in any order, to convert an oxidation product into a compound which can react with Gibbs reagent.

In a preferred embodiment of the invention, test oxidation enzymes are provided by host cells which have been transformed by genetic engineering techniques, so that they express the test oxidation enzyme. The test enzyme can be produced and retained inside the cell, or it can be secreted outside the cell. In either case, test enzyme can be recovered from host cells for use in an in vitro, solid-phase, or liquid-phase, assay, where the enzyme is combined with the other assay ingredients. Enzyme that is secreted outside the cell can usually be recovered in a non-destructive manner, by collecting it from the growth medium, usually without disrupting the cells, or on a plate where the cells are grown. When the enzyme remains inside the cell, it is typically recovered by breaking open the cells so that the enzyme can be released and separated from the medium and cell debris. Particularly preferred applications of the invention include screening methods for identifying or evaluating which of a multitude of mutated or evolved enzymes displays the best oxidation activity under selected conditions.

A schematic representation of the chemical reactions applied in a preferred embodiment of the screening invention is shown in FIG. 14. An oxidation enzyme, in this case a dioxygenase, oxidizes a substituted aromatic compound, in this case into a cis-dihydrodiol. Generally, the oxygenated product is formed by the addition of one or more hydroxyl (OH) groups at one or more ring positions of an aromatic, cyclic, or heterocyclic substrate, e.g., in place of hydrogen. With respect to the substrate, the “X” substituent of the benzene ring may be, for instance, a halogen, hydroxyl, t-butyl, or hydrogen, in which cases the substrate is halogenated benzene, toluene, t-bytul benzene, and benzene, respectively. Other potential substrates include biphenyl, heterocyclic compounds such as 4-picoline, and various other mono- and di-substituted monocyclic aromatics. The oxygenated products are usually difficult to detect or measure directly. Therefore, according to the methods of the invention, the oxygenated product is converted into a compound which can be readily detected. For example, using route B, the oxidation product, in this case a cis-dihydrodiol, is converted into a phenol by acidification with HCl. The phenol is then reacted with Gibbs reagent to form a detectable composition, in this case, having blue color. Alternatively, using route C, the cis-dihydrodiol is converted into a catechol by dehydrogenase in the presence of the coenzyme NAD⁺. Next, the catechol is reacted with Gibbs reagent to form a detectable compound.

Liquid-phase Assay

In one preferred embodiment, the so-called “liquid-phase assay”, a test enzyme is contacted with the substrate and an oxygen donor in a liquid, typically an aqueous solution. This type of assay is advantageously performed using microtiter plates. The test oxidation enzymes are provided directly into each well, or provided by arraying individual host cells expressing the test enzyme into each well. Next, one of the following two routes are taken to convert at least one product into a compound which is easily detectable by a subsequent Gibbs assay. The first route involves acidification of the liquid so that the product is converted into a phenol. For an oxidation product such as a cis-dihydrodiol, the pH may be lowered to below 4, or to about 2.5. Alternatively, product in a neutral pH buffer may be mixed with an equal amount of 0.1M HCl to facilitate oxidation/dehydrogenation into a phenol. The second route comprises the presence of a dehydrogenase enzyme to convert the product into a catechol. For an oxidation product such as a cis-dihydrodiol, the enzyme cis-dihydrodiol dehydrogenase is advantageously used. The dehydrogenase enzyme may be added together with the oxidation enzyme, or may be added after initiation or termination of the first oxidation reaction of the substrate. In another embodiment, it is not necessary to recover test enzyme from host cells, because the host cells expressing the oxidation enzyme are used directly in the screening method

Solid-phase Assay

A preferred embodiment of the methods of the invention is the so-called “solid-phase assay”. In this embodiment, colonies of host cells are screened directly without individually arraying the clones into microtiter plates, by use of digital imaging tools. The hosts cells are typically spread or streaked, using any method known in the art, onto a solid support such as agar plates to allow colonies to form. Next, the host cells are contacted with a substrate. Substrates and oxygen donors typically cross the cell membrane and enter the cell. If so, the substrate and donor encounter the test enzyme. In a preferred embodiment, the host cells are first transferred to another solid support or transferring device such as a membrane, e.g., a nitrocellulose membrane, which is then contacted with or exposed to a substrate. For example, the transferring device or membrane can be placed in a chamber containing the substrate in vapor phase. The product formed is thereafter either directly contacted with Gibbs reagent, or converted into compounds which are more effectively detected by the subsequent Gibbs assay. This can be accomplished using either of the two routes described for the liquid assay, which result in the product being converted to compounds such as, but not limited to, phenols or catechols. In a preferred embodiment, the dehydrogenase enzyme, most preferably cis-dihydrodiol dehydrogenase, is introduced into the assay by transforming the host cells with a plasmid which encodes for the dehydrogenase enzyme and expressed and retained within the cells. Even more preferably, the dehydrogenase gene is included in the plasmid used to transform the cells with the oxidation enzyme. Alternatively, though less desirable, the dehydrogenase enzyme itself may be added directly to the host cells before or during the screening assay. The oxygenated products resulting from the above reaction steps may be reacted with intracellular Gibbs reagent, or allowed to cross the cell membrane (leave the cell) and react with Gibbs reagent outside the cells, to form a detectable reaction product. Though less desirable, any assay component which does not cross the cell membrane may be introduced directly to the interior of the cell by known means.

In a particularly preferred embodiment, host cells are transformed to produce both a test enzyme and a dehydrogenase enzyme. Substrate and donor are contacted with the cells by adding a solution containing the two, or by providing the added to the cell medium and are taken up by the cells. Active enzyme produces an oxygenated substrate, which is converted to a detectable reaction product by the coupling enzyme.

The solid-phase assay is a high-throughput (10,000 clones/day) screen for dioxygenase activity by which thousands or tens of thousands of host cell clones can be screened per day. The cis-dihydrodiol product of dioxygenase bioconversion is converted to a phenol by acidification or to a catechol by reaction with cis-dihydrodiol dehydrogenase. Gibbs reagent reacts quickly with these oxygenated aromatics to yield colored products that are quantifiable using a microplate reader or by digital imaging and image analysis. The method is reproducible and quantitative at product concentrations of only 30 μM, with essentially no background from media components. This method is an effective general screen for aromatic oxidation and should be a useful tool for the discovery and directed evolution of, e.g., oxygenases (see below).

The Gibbs assay is particularly suited for the solid-phase assay since it produces a signal that can be observed from outside the cell by visual inspection, spectrometric measurements, or digital imaging. Such measurements are non-destructive, and allow for isolation and further work with cells that produce active enzymes. Transformed host cells that produce more active oxidation enzymes “light up” in the assay and can be readily identified, and distinguished or separated from cells which do not “light up” as much and which produce inactive enzymes, less active enzymes, or no enzymes.

Oxygenated substrate that is secreted by the cell can interact with extracellular Gibbs reagent to form a detectable reaction product. If the host cells are grown on a solid support, a light signal may be identifiable as a ring which “lights up” around cells which product active oxidation enzyme. Depending on how close together neighboring cells are growing, this method may allow for active and non-active host cells to be distinguished.

In embodiments where all of the host cells in or on a particular medium are producing the same test enzyme, the choice of intracellular or extracellular approach is likely to be determined as a matter of convenience, unless other circumstances favor or require one technique over the other.

### Test Enzymes

The oxidation enzymes for screening according to the methods of the invention can be members of enzyme libraries, produced by, e.g., random mutagenesis, DNA shuffling, or any other technique useful for directed evolution. In another preferred embodiment, the oxidation enzymes are members of expressed gene libraries.

Suitable oxidation enzymes which may be used include chloroperoxidase (CPO), cytochrome P450 enzymes, methane monooxygenases (MMO), toluene monooxygenases, toluene dioxygenases (TDO), biphenyl dioxygenases and naphthalene dioxygenases (NDO), any of the many mono- and di-oxygenases, or any other oxidation enzyme exemplified herein. Specific examples include chloroperoxidase, methane monooxygenase, toluene monooxygenase, naphthalene dioxygenase, biphenyl dioxygenase, phenol hydroxylase, dehalogenase, microperoxidase, cytochrome P450.

Because of their broad substrate range and high enantioselectivity, dioxygenases are particularly preferred for directed evolution using the screening methods of the invention (Gibson and Parales (2000); Boyd et al. (1998)). Bacterial dioxygenases are multicomponent enzyme systems that catalyze the stereospecific introduction of molecular oxygen into a wide variety of aromatic compounds to form arene cis-diols (FIG. 14). In the first step of the dioxygenase mechanism, electron transfer proteins shuttle electrons from NADH to the Reiske [2Fe-2S] cluster of the terminal dioxygenase (Butler and Mason (1997)). These electrons activate the mononuclear iron at the active site of the enzyme, allowing molecular oxygen and substrate to bind and react (Gibson and Resnick (1996:3)). More than 300 diverse substrates ranging from halogenated ethylenes (Lange and Wackett (1997) to polyaromatic hydrocarbons such as phenanthrene and dibenzo-1,4-dioxin (Gibson and Resnick (1996:3); Gibson and Parales (2000)) can be dihydroxylated by dioxygenases. In the natural aromatic biodegradation pathway, dihydroxylation is followed by rearomatization to form a catechol by cis-dihydrodiol dehydrogenase. Catechol is further degraded to provide a carbon and energy source for the host organism.

Dioxygenases are excellent candidates for applications in bioremediation (Wackett (1995)), synthetic chemistry (Sheldrake (1992)) and combinatorial biocatalysis (Wendeborn et al. (1998)). Optically pure arene cis-diols formed by recombinant organisms lacking cis-dihydrodiol dehydrogenase have several proposed applications as starting materials in the synthesis of chiral drugs and specialty chemicals (Sheldrake (1992)). Dioxygenase bioconversions are limited, however, by the low activity toward unnatural substrates, the low stability, especially in vitro, the low solubility and toxicity of their substrates, the NADH cofactor requirement, and by product inhibition and toxicity (Jenkins et al. (1986)); Wahbi et al. (1996); Wilkinson et al. (1996); and Harrop et al. (1992)). For many prospective applications, it will be necessary to improve the catalyst. This can be done using directed evolution, in which cycles of mutagenesis, recombination, and screening are used to accumulate mutations needed (Kumamaru et al. (1998); and Bruhlman and Chen (1998)). A reliable, high throughput activity assay is crucial to any directed evolution effort. Rapid screens ideally employ whole-cell conversions, with little or no purification of products.

As described herein, the methods of the invention also provide for screening methods for thioesterases and enzymes which catalyze the hydroxylation of phenyl ethers.

Host Cells and Vectors

In one aspect of the invention, a whole cell screening method is provided, in which a test oxidation enzyme is produced by a transformed host cell using a suitable expression system. The types of host cells and expression systems which are suitable for use in accordance with the invention are those which are capable of expressing oxidation enzymes. Host cells which can also express coupling enzymes are preferred. E. coli is one preferred exemplary cell. Other exemplary cells include other bacterial cells such as Bacillus, Pseudomonas, yeast cells, insect cells and filamentous fungi such as any species of Aspergillus cells. For some applications, such as screening for toxicity of certain compounds, plant, human, mammalian or other animal cells may be preferred.

Suitable host cells may be transformed, transfected or infected as appropriate by any suitable method including electroporation, CaCl₂ mediated DNA uptake, fungal infection, microinjection, microprojectile transformation, viral infection, or other established methods. Appropriate host cells include bacteria, archaebacteria, fungi, especially yeast, and plant and animal cells. Of particular interest are E. coli, and Saccharomyces cerevisiae.

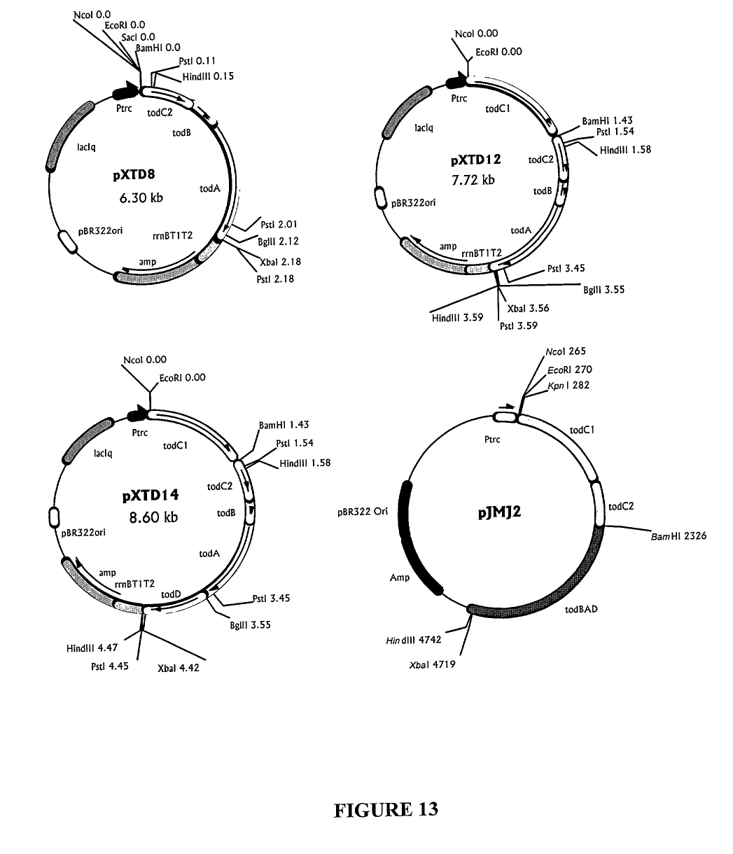

Any of the well-known procedures for inserting expression vectors into a cell for expression of a given peptide or protein may be utilized. Suitable vectors include plasmids and viruses, particularly those known to be compatible with host cells that express oxidation enzymes or oxygenases. Examples of vectors that contain genes for oxidase enzymes are provided in the Examples and in FIG. 13.

The invention is especially well suited for screening large numbers of mutant oxygenases wherein cells are transformed with a number of different vectors which express different mutant oxygenases. The mutant oxygenase genes can be prepared using procedures such as DNA shuffling, as shown for example in U.S. Pat. No. 5,605,793 or by random mutagenesis, for example using error prone polymerase chain reactions (PCR). See, e.g., U.S. Pat. Nos. 5,741,691 and 5,811,238 and WO Publication No. 98/42832.

Once the host cell has been transformed with the desired vector expressing the oxidation enzyme(s) or oxygenase to be tested, the cell line is maintained and grown under conditions which promote expression of the oxygenase within the cell. In general, the oxygenase remains within the cell and is not excreted. After the transformed cells have been cultured for a sufficient time to generate oxygenase, the cells are contacted with or otherwise treated with the substrate of interest, or the oxygenase harvested from the cells and contacted with substrate in vitro. Preferred cells for these applications are bacterial cells such as E. coli and Bacillus, and yeast cells, e.g., S. cerevisiae, in which libraries of different mutants (dozens or more, and typically thousands) can be made.

Although it is possible to add dehydrogenase enzyme for reaction with oxygenated compound that is produced by the oxygenase, it is preferred that the dehydrogenase enzyme be co-expressed within the cells to provide an intracellular screening system. The transformation of the cell to express the dehydrogenase enzyme is accomplished in a manner similar or analogous to transforming the cell to express the oxygenase. The result is a cellular system which provides for the indirect detection of the presence of oxygenated compounds which are produced within the cell when a substrate is reacted with an oxygenase expressed within the cell. The co-expression of the dehydrogenase enzyme provides a readily available source of enzyme to convert the oxygenated compound to form colored products together with Gibbs reagent.

### Application to Other Enzymes and Reactions

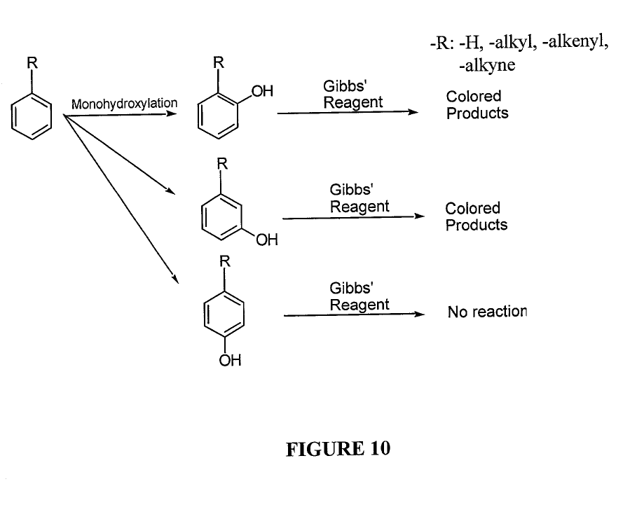

The methods described above may also be useful in diagnosing or quantifying the products of other classes of enzymatic reactions. The powerful solid-phase screening technique discussed earlier may be applied to the determination of monooxidation reactions. These reactions result in phenolic derivates, which often couple with Gibbs' reagent to form colored products, as shown in FIG. 10. The reaction pathway is conceptually simpler to implement than the assay for dioxygenase activity, because the enzymatic reaction leads to hydroxylated products which directly form with the Gibbs' reagent a colored product.

A variety of ortho- and meta-substituted phenols readily couple to Gibbs' reagent whereas para-substituted phenols do not react or do not react efficiently with Gibbs' reagent (Quintana et al. (1997)). The solid-phase method could be used to assay colonies expressing candidate monooxygenases.

This assay system can be extended further to assaying for activity towards polycyclic aromatic compounds such as naphthalene, anthracene or benzpyrene. After the hydroxylation, these compounds will form a similar color with the Gibbs reagent as the phenolic compounds.

Enantioselectivity

Because the stereochemistry of cis-dihydrodiols produced by dioxygenases is often crucial in the context of industrial applications, it would be desirable to be able to screen dioxygenase candidates for enantioselectivity. Thus, the Gibbs' assay could be used to determine the enantiomeric excess of cis-dihydrodiols produced by dioxygenases. The chiral dioxygenase product is a dihydrodiol, with two chiral centers, and therefore four theoretically possible enantiomers. Since no prokaryotic dioxygenases produce trans-dihydrodiols, but some dioxygenases do make racemic cis-dihydrodiol, it is likely that genetic variants will also only produce cis-dihydrodiol (Drew et al. (1999); Boyd et al. (1998); Gibson and Resnick (1996:2)).

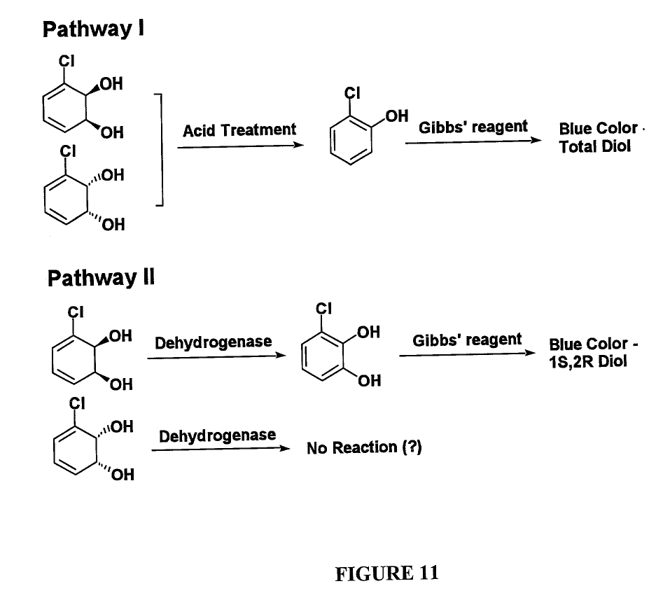

FIG. 11 shows a proposed scheme for determining the enantiomeric excess of a mixture of cis-dihydrodiol enantiomers. The scheme relies on the high enantioselectivity of the 2,3-dihydrodiol dehydrogenase from toluene dioxygenase, which has been documented using indene as the initial substrate (Drew et al. (1999)). Two assays are run in parallel: one detects the total amount of dihydrodiol present (pathway I), and the other uses the enantioselective dehydrogenase to measure only the amount of one cis-dihydrodiol enantiomer (pathway II).

The enantioselectivity of the dioxygenase enzyme could be determined by comparing the two final measurements from the two pathways. The exact enantioselectivity of the dehydrogenase toward the diol of interest would be required to calibrate the assay. To determine this, a racemic mixture of dihydrodiol, possibly produced by chemical oxidation by OsO₄, could be biotransformed by toluene cis-dihydrodiol dehydrogenase. Chiral chromatography could then be used to determine the enantioselectivity of the dehydrogenase.

Because a different combination of enzymes is required for both pathways, this assay would be done in microwell plates. Since two measurements are subtracted to obtain the enantioselectivity, the accuracy of this measurement would be lower than each activity measurement, and small losses (10-20% ee) in enantioselectivity may not be detected.

Phenyl Ethers

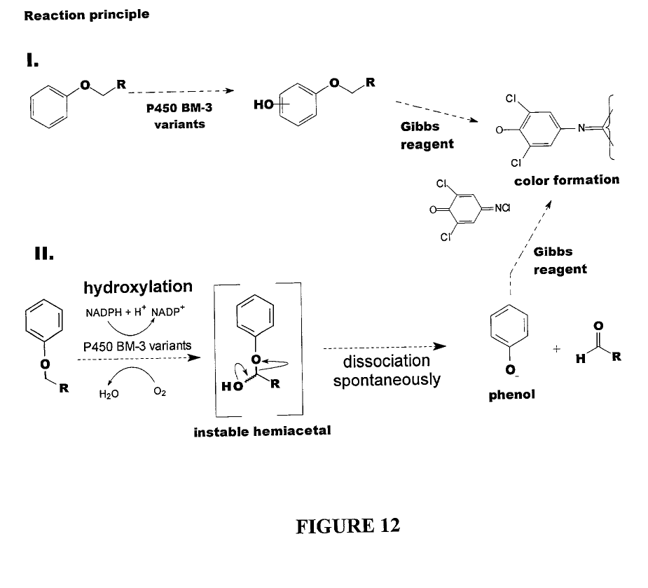

Oxidation of phenyl ethers is another class of reactions that can be monitored using Gibbs' reagent (FIG. 12). The product of such an oxidation reaction can be a hydroxylated phenyl ether or a phenol ether, including substituted derivatives of the foregoing.

Biocatalytic ether cleavage proceeds by hydroxylation of the terminal aliphatic carbon of the side chain, followed by spontaneous dissociation resulting in a phenol and an aldehyde (reaction II). The hydroxylation can also occur at the aromatic part of these substrte class also resulting in phenol ether derivates which also form a color with the Gibbs reagent (reaction I). The pathway shown in the FIG. 12 may be especially effective for discovery of novel oxidative enzymes, since both aromatic monohydroxylation and phenol ether cleavage activities can be assayed simultaneously. It is further possible to distinguish between both reaction types by employing the hydroxylation reaction of the phenoxy- and corresponding phenyl-derivates on two separated solid phase matrices. For instance, only a hydroxylation at the aromatic part of the substrate will lead to a color formation with the Gibbs reagent in both reactions.

The screening methods described above are applicable to dioxygenase bioconversions of a number of aromatic substrates. These methods may also find application in screening for other types of aromatic oxidation, such as monooxidation, dealkylation, and oxidative dehalogenation, that also result in phenolic products. Quintana et al. (1997) describe 14 phenols and catechols that react with Gibbs reagent to yield colored products. In general, most phenols with a good leaving group at the para position (—H, —OCH₃, halogens) will couple readily with Gibbs reagent and have absorbance maxima in the range 500-700 nm (Josephy et al. (1984)).

Sulfhydryl Groups

Oxidation of aromatic or aliphatic thioester-containing compounds is a further class of reactions that can be monitored using Gibbs' reagent (see below). The product of such an oxidation reaction, catalyzed by, e.g., a thioesterase, is a carboxylic acid-containing moiety and a thiol-containing compound, of which the thiol-containing compound can be detected using the Gibbs reaction.

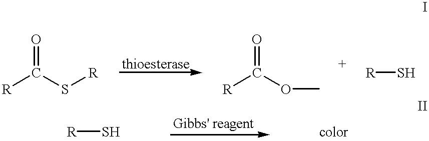

Examples of practicing the invention are provided, and are understood to be exemplary only, and do not limit the scope of the invention or the appended claims. A person of ordinary skill in the art will appreciate that the invention can be practiced in many forms according to the claims and disclosures here.

### EXAMPLE 1

Liquid-phase Screening Methods for Determining Dioxygenase Activity

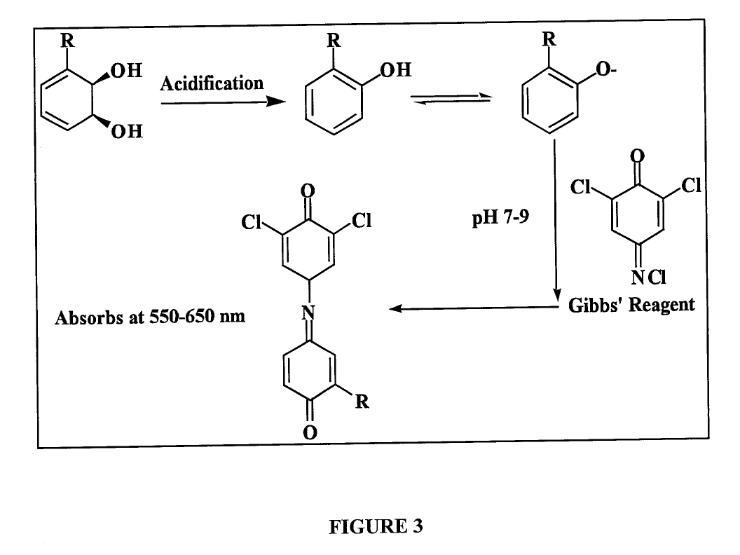

This Example describes an improved method for dioxygenase screening. The vicinal cis-dihydrodiols of aromatic or heterocyclic compounds were effectively converted to the corresponding phenols or hydroxypyridines by acidification, or catechols or dihydroxypyridines by enzymatic reduction, which allowed for the formation of more intense color by the following Gibbs assay. The Gibbs assay with acidification process is outlined in FIG. 3. Thus, each biocatalyst could be evaluated more easily and precisely. Moreover, the principles were successfully applied to the screening of dioxygenase libraries.

Expression Systems

Four expression plasmids were used (FIG. 13). All plasmids contained an ampicillin resistance gene and the ptrc promoter for high expression induced by IPTG (Amann et al. (1988)). For expression of these plasmids, the E. coli strain BL21(DE3) was used. Toluene dioxygenase was first discovered in Pseudomonas putida F1 and was overexpressed in E. coli by Gibson et al. (1989).

The plasmid, pXTD12 was designed for the random mutagenesis of toluene dioxygenase from Pseudomonas putida F1, which is composed of ISP_{TOL} α subunit, β subunit, Ferredoxin_{TOL} and Reductase_{TOL} encoded by todC1, C2, B and A gene, respectively. pXTD12 was constructed by the introduction of todC1 into a cloning vector pXTD8 carrying todC2BA, and used for the expression of both wild type and mutant enzymes.

Another plasmid, pXTD14, contained all the genes of pXTD12, and also toluene cis-dihydrodiol dehydrogenase (todD).

pJMJ2 was almost identical to pXTD14, but had restriction sites designed for simultaneous evolution of both subunits of ISP_{TOL}.

pDTG601 contained the genes encoding for the multicomponent toluene dioxygenase system.

Screening Method for Toluene Dioxygenase Mutant Library

A toluene dioxygenase screening method was developed, comprising the following steps:

1. Transform a plasmid, such as pXTD12, into E. coli BL21(DE3) by the calcium method, and plate an appropriate volume of the transformation mixture onto a LB-100A agar plate.

2. Incubate at 30° C. until colonies become discernible.

3. Fill each well of a 96-well plate with LB-100A and innoculate each well with a different transformant.

4. Shake the plate at 270 rpm, at 37° C., for 12-18 hours.

5. Transfer 10 μl of the resulting culture to 100 μl of M9-GIA, and mix thoroughly.

6. Incubate at 30° C. for 5 hours without shaking.

7. Combine 20 μl of culture with a 80 μl of a reaction medium containing 50 mM sodium phospate buffer (pH 7.4), 0.2% glucose and 6.25 mM toluene. Mix thoroughly.

8. Incubate at room temp. for 30 minutes.

9. Add a 100 μl of 0.1 M HCl to the reaction mixtures.

10. Centrifuge at 3,000 rpm for 5 minutes.

11. Transfer a 100 μl aliquot of supernatant to an empty well.

12. Incubate the plate at 37° C. for 30 to 40 minutes.

13. Add 20 μl of 1 M Tris-HCl (pH 8.5) to the supernatant and mix well.

14. Add 25 μl of 0.4% Gibbs' reagent in ethanol.

15. After a 5 minute incubation at room temperature, measure the absorbance at 590 nm.

LB-100A is LB medium supplemented with 100 mg/L Ampicillin. M9-GIA is M9 medium containing 0.4% glucose, 0.5 mM IPTG and 100 mg/L Ampicillin.

The above screening method was applied, in 96-well plate format, for both wild-type toluene dioxygenase and toluene dioxygenase mutants. In similar experiments, chlorobenzene and 4-picoline were applied as substrates instead of toluene. As with toluene, these substrates were added to a final concentration of 5 mM.

Results

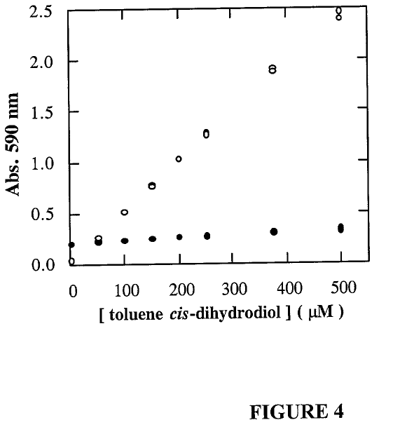

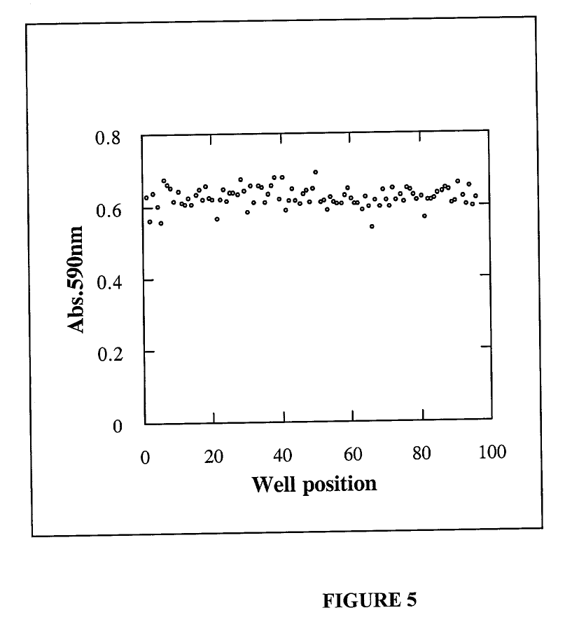

Like the other cis-dihydrodiols, toluene cis-dihydrodiol was dehydrated more effectively in the acidic condition (^{˜}pH 2.5) than in the neutral condition. Accordingly, following neutralization of the reaction mixture, the Gibbs assay with acidification was found to be a more sensitive measurement of toluene cis-dihydrodiol than the Gibbs assay without acidification, as shown in FIG. 4. When 96 transformants carrying wild-type toluene dioxygenase were screened using this method, the resulting measurements, shown in FIG. 5, were uniform with a standard deviation of only 4.2%. This shows that a low rate of false positives would be encountered during the screening of a library.

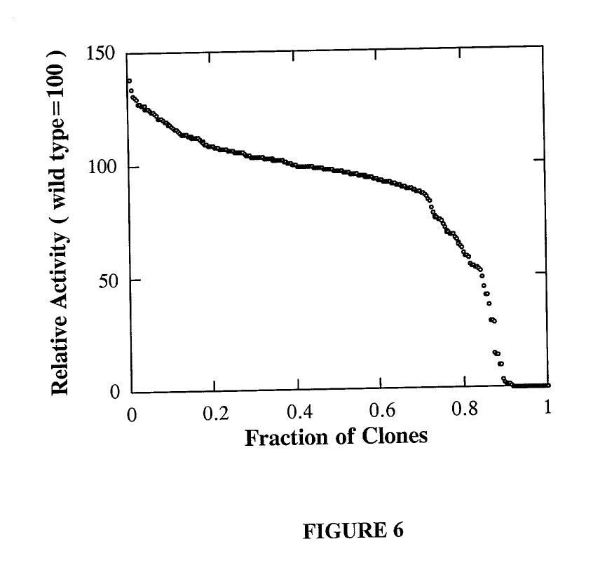

A mutant library of toluene dioxygenase was generated by the random mutagenesis of todC1 gene. Approximately 800 transformants expressing mutant todC1 genes were screened in 96-well plate format using the method described above. FIG. 6 shows the relative activity of each mutant toluene dioxygenase toward toluene. The best mutant was chosen and proven to have 1.3 times higher activity toward toluene than the wild-type enzyme.

The above procedure was applied, also in a 96-well microplate format, to measure dioxygenase activity toward chlorobenzene. The detection limit of this assay was found to be 10 μM chlorobenzene cis-dihydrodiol. When 96 wild-type TDO cultures (BL21(DE3) expressing pXTD12) were assayed, the distribution of activity measurements was approximately normal with a standard deviation of 7.4%. Therefore, this screen for dioxygenation of chlorobenzene should obtain only a low rate of false positives when applied to a mutant library.

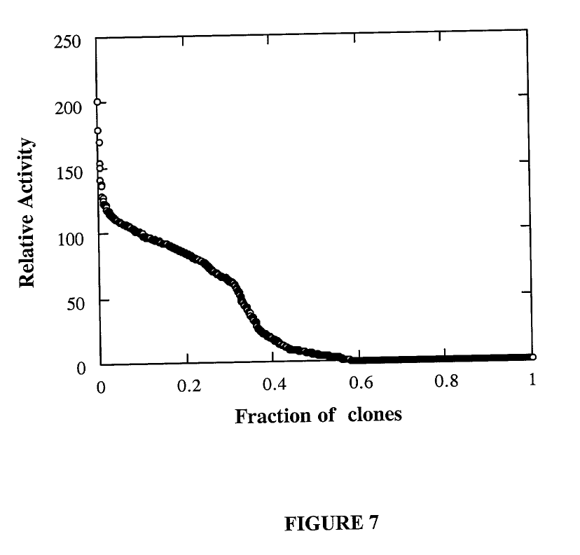

This screening method was also successfully applied to measure wild-type toluene dioxygenase activity toward 4-picoline. The mutant library was also screened for variants having a higher dioxygenase activity toward 4-picoline. Five mutants were identified using the screening method which showed more than 1.5 times higher activity toward 4-picoline than the wild-type enzyme (FIG. 7).

Discussion

The microplate assay described above is applicable to a wide variety of substrates and reactions. Deletion of the acidification step in FIG. 3 would lead to an assay for monooxidation or oxidative dealkylation. All of the manipulations are amenable to robotic automation. The assay can also be modified by coexpression of cis-dihydrodiol dehydrogenase, as shown below. This modification may be useful for the quantitation of cis-dihydrodiols that do not acidify readily to the corresponding phenol (such as chlorobenzene cis-dihydrodiol). Using either the dehydrogenase method or the acidification method, dioxygenase activity toward an array of substrates can be quantified, including halogenated benzenes, biphenyl, heterocyclic compounds such as 4-picoline, and a variety of mono- and di-substituted monocyclic aromatics, such as toluene, benzene, and t-butyl benzene.

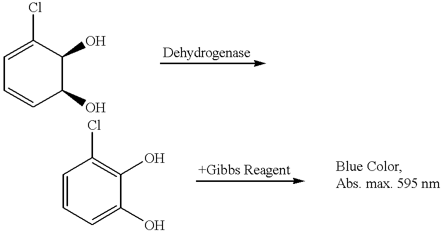

### EXAMPLE 2

Solid-phase Screening Methods for Determining Dioxygenase Activity

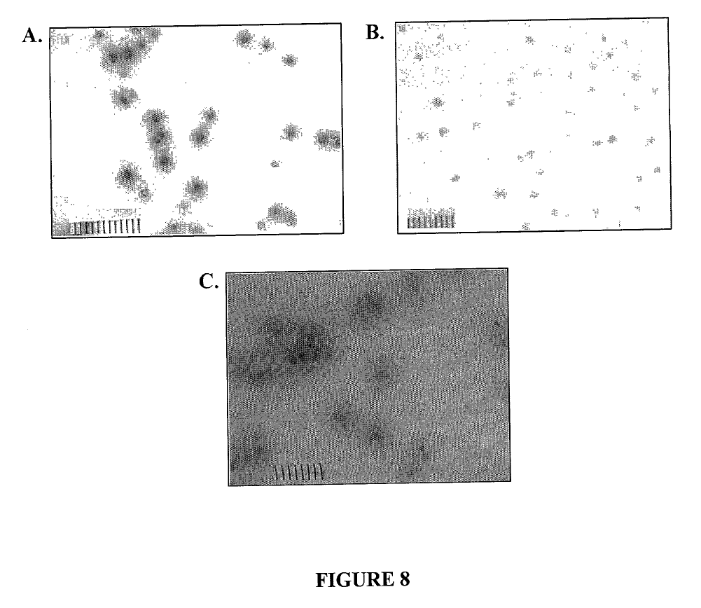

The rate-limiting step of the microplate assay described in Example 1 is innoculation of clones into 96-well microplates. To attain higher throughput, the following method was developed for applying the chemistry of the Gibbs' assay directly to freshly transformed colonies. In this solid-phase method, colonies were transferred using a nitrocellulose membrane to a chamber containing the substrate of interest in the vapor phase. In a modification of the method, dehydrogenase was co-expressed. The color was developed by contact with an agar plate containing Gibbs' reagent (FIGS. 8A and 8B). The detailed procedure for this method is shown below.

Materials

Terrific broth mix, Gibbs' reagent, ampicillin, chlorobenzene, and toluene were supplied by Sigma. IPTG was supplied by ICN Biomedicals Co. The nitrocellulose membranes used had a pore size of 0.45 μm, and a diameter of 132 mm, and were supplied by Schleicher & Schuell. Bacto-Agar was supplied by Difco, and agarose was supplied by Gibco. 150 mm×15 mm petri dishes were supplied by Falcon.

Method I: Detection of Catechol Formed by Dehydrogenase

A toluene dioxygenase screening method was developed, comprising the following steps:

1. Transform BL21 (DE3) Gold competent cells with a plasmid coding both for a dioxygenase enzyme, and its corresponding dehydrogenase. Plasmids such as pJMJ2 from FIG. 13 are suitable.

2. Spread freshly transformed cells on terrific broth agar plates containing 100 mg/L Ampicillin and 0.5 mM IPTG. 150×15 mm petri dishes were used throughout the assay.

3. Incubate at 37° C. for 6 hrs, then at 30° C. for 12 hrs.

4. Place a nitrocellulose membrane on seed petri dish, allow to wet for 5 minutes. Transfer cells to a M9 minimal medium (48) plate, also containing 1.6% glucose, 100 mg/L ampicillin, and 4% bacto-agar, with the cells facing up.

5. Suspend petri dish in an airtight container. In the container, place a dish containing 1 mL of the aromatic substrate of interest. Incubate for 20 minutes at 30° C.

6. Melt 1.8 g agarose in 60 mL of water. After cooling, add 0.75 mL of 2% Gibbs' reagent dissolved in ethanol. Pour into one petri dish, and allow hardening and cooling with the lid on.

7. Remove the membrane and put it cell-side-up on the Gibbs reagent plate.

Method II: Conversion to Phenol, and Subsequent Detection

In a modification of the above method, dehydrogenase was not coexpressed, but the cis-dihydrodiol was acidified by the vapor from 9.5M HCl to form a phenol which also reacts with Gibbs' reagent (FIG. 8C). The procedure comprises the following steps:

1. Transform BL21 (DE3) Gold competent cells with a plasmid coding for a dioxygenase enzyme system, without the dehydrogenase gene. pXTD12 (see FIG. 13) is suitable.

2. Spread freshly transformed cells on terrific broth agar plates containing 100 mg/L Ampicillin and 0.5 mM IPTG. 150×15 mm petri dishes are used throughout the assay.

3. Incubate at 37° C. for 6 hrs, then at 30° C. for 12 hrs.

4. Place a nitrocellulose membrane on seed petri dish, allow to wet for 5 minutes. Transfer cells to a M9 minimal medium (70) plate, also containing 1.6% glucose, 100 mg/L ampicillin, and 4% bacto-agar, with the cells facing up.

5. Suspend petri dish in an airtight container. In the container, place a dish containing 1 mL of the aromatic substrate of interest. Incubate for 20 minutes at 30° C.

6. Remove dish containing the substrate, and replace with a dish containing 20 mL of freshly prepared 9.5 M HCl. Incubate sealed container for 15 minutes at 37° C. The pH of the surface of the plate should be about 2, as measured by gently pressing a strip of 0-6 pH paper against a colony-free area of the membrane.

7. Melt 1.8 g agarose in 60 mL of 50 mM phoshate buffer, pH 7.4. After cooling, add 0.75 mL of 2% Gibbs' reagent dissolved in ethanol. Pour into one petri dish, and allow hardening and cooling with the lid on.

8. Remove the membrane and put it cell-side-up on the Gibbs' reagent plate.

Results

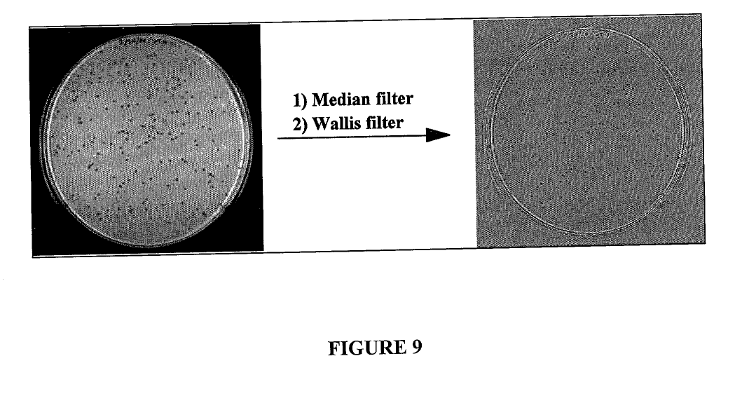

Results from the solid-phase assay conducted both according to Method I and Mehtod II are shown in FIG. 8. Relative activity was quantified by digital imaging with a Bio-rad Fluor-S™ Multiimager, followed by image analysis using the program Optimas. Under reproducible lighting conditions and camera settings, an approximate 1300×1000 pixel 8-bit gray-scale image was obtained using the Multiimager. In Optimas, this raw image was filtered with a median filter, followed by a Wallis filter. This eliminated any large-scale contrast across the image, as shown in FIG. 9.

The Wallis filter adjusted the average gray level of the image to 128 (on the 8-bit 0 to 256 scale). A threshold was set manually at approximately 115 to eliminate the background. Isolated areas with gray levels less than 115 corresponded to colonies, and were detected by Optimas. The pixel intensities inside each area were averaged to obtain the mean gray value for each colony. Other statistics provided by Optimas, such as the colony area and circularity, can be used to eliminate colonies that overlap. The mean gray value of each true colony was subtracted from the mean gray of the background (128) to obtain a quantitative assessment of the color change. TABLE 1 shows how the average color change of wild-type colonies increased with longer exposure times to substrate vapor. As shown in TABLE 1, the standard deviation of the color change measurements was low, showing a low false positive rate for screening of a genetically diverse population.

TABLE 1
Results of solid-phase assay using different biotransformation times

| Biotransformation | Average Color | % Standard | # of Colonies |
|---|---|---|---|
| Time (min). | Change¹ | Deviation | Sampled |
| 10 | 15.3 | 8.19% | 86 |
| 15 | 18.1 | 7.02% | 133 |
| 20 | 28.3 | 6.21% | 163 |
| 37 | 32.5 | 5.22% | 131 |

¹Average color change was calculated from a filtered, gray-scale digital image of the colonies. Pixel intensities ranged from 0 to 256. The color change of each colony was calculated by averaging the intensity of its pixels, and subtracting the average intensity of the regions of the plate containing no colonies.

### EXAMPLE 3

Liquid and Solid-phase Screening Methods for Determining Dioxygenase Activity

This Example presents a high-throughput screen for dioxygenase activity. In this method (see FIG. 14), the arene cis-diol products of the dioxygenase are either converted to phenolic compounds through acidification or converted to catecholic compounds through further reaction with cis-dihydrodiol dehydrogenase, which is coexpressed with the dioxygenase. This step is followed by colorimetric detection with 2,6-dichloro-p-benzoquinone (Gibbs reagent). Specifically, toluene dioxygenase enzymes are screened using the modified Gibbs assay of the invention, and chorobenzene is used as the oxidation substrate.

This versatile method can be applied to numerous aromatic dioxygenase substrates and can be used as a general screen for aromatic oxidation reactions. A solid-phase version of the method was developed to screen bacterial colonies directly without individually arraying the clones into microtiter plates using digital imaging tools. This sensitive and reproducible screening method advantageously applied for directed evolution and catalyst discovery.

Materials and Methods

Terrific Broth, ampicillin, chlorobenzene, and Gibbs reagent were purchased from Sigma (St. Louis, Mo.). Isopropyl-β-D-thiogalactopyranoside (IPTG) was purchased from ICN Biomedicals, Inc. (Aurora, Ohio). ME25 0.45 μm nitrocellulose membranes were purchased from Schleicher & Schuell (Keene, N.H.). V-bottom microtiter plates were purchased from Corning (Corning, N.Y.). BL21 (DE3) cells were provided by Stratagene (La Jolla, Calif.). Taq buffer, MgCl₂, and AmpliTaq DNA polymerase were supplied by Perkin Elmer (Norwalk, Conn.). Cis-(1S,2S)-chloro-3,5-cyclohexadiene-1,2-diol (chlorobenzene cis-dihydrodiol) was purchased from QuChem (Belfast). T4 DNA ligase was purchased from Boeringer Manheim (Germany). Restriction enzymes were purchased from New England Biolabs (Beverly, Mass.). The genes encoding toluene dioxygenase were kindly provided by D. T. Gibson on the plasmid pDTG602 (Zylstra and Gibson (1989)).

Liquid-phase Screening for Activity Toward Chlorobenzene

Colonies of E. coli BL21 (DE3) expressing pJMJ8 (See TABLE 2 below) were inoculated into the wells of a 96-well plate containing 100 μL of Luria-Bertani (LB) medium (Sambrook (1989)) supplemented with 100 mg/L ampicillin. Cells were grown for 18 hours in a shaking incubator set to 37° C. 5 μL of each culture was transferred to a V-bottom microtiter plate containing 95 μL of M9 media (Sambrook (1989)) supplemented with 100 mg/L ampicillin, 1 mM IPTG, 1.6% D-glucose, and 80 mg/L FeSO₄-7H₂O (M9-GIA). These cultures were incubated at 30° C. for 5 hours without shaking. To start the biotransformation, 50 μL of M9-GIA also containing 15 mM chlorobenzene was added, and the plate was wrapped in Saran wrap and incubated at 30° C. for 90 minutes. The 96-well plate was then centrifuged at 1700×g for 10 minutes. 100 μL of supernatant was transferred to a flatbottom, transparent microtiter plate containing 100 μL of 0.1M HCl. This plate was incubated at 37° C. for 30 minutes, and then 20 μL of 1M Tris-HCl, pH 8.5, was added to raise the pH. At this point, 25 μL of 0.4% Gibbs reagent in ethanol was added to each well. For the chlorobenzene assay, the absorbance at 652 nm was read after 40 minutes at room temperature.

Solid-phase Screening for Activity Toward Chlorobenzene

Plasmid pJMJ2 was transformed into BL21(DE3) competent cells according to manufacturer's instructions and plated on terrific broth (TB) agar plates containing 100 mg/L ampicillin and 0.5 mM IPTG. Plates were incubated for 6 hours at 37° C., and then at 30° C. for 12-14 hours. Colonies were lifted with a 132 mm diameter nitrocellulose membrane and transferred to M9 media (Sambrook et al. (1989)) containing 4% agar, 100 mg/L ampicillin, 1.6% D-glucose, and 80 mg/L FeSO₄-7H₂O. The colonies were then incubated for 20 minutes in an airtight container at 30° C. containing an open dish of chlorobenzene. The membrane was transferred to a 4% agarose plate also containing 0.025% Gibbs reagent (added as a 2% solution in ethanol).

TABLE 2
Plasmids used for expression of toluene dioxygenase cistron

| Plasmid | | | |
|---|---|---|---|
| Name | Gene Insert/Vector | Promoter | Description |
| pJMJ2 | todC1C2BAD/ptrc99A | Ptrc | Expresses toluene |
| | | | dioxygenase (todC1C2), |
| | | | electron transfer proteins |
| | | | (todBA) and dehydrogenase |
| | | | (todD) |
| pJMJ8 | todC1C2BA/ptrc99A | Ptrc | Expresses toluene |
| | | | dioxygenase (todC1C2), |
| | | | and its electron transfer |
| | | | proteins (todBA) |

Digital Imaging and Analysis

The membrane in the final agarose plate was imaged using a Fluor-S MultiImager (Biorad, Hercules, Calif.) equipped with a Tamron SP AF20-40 mm lens (Tamron Co., Ltd., Tokyo, Japan). Digital images (1300×1000 pixels) were imported to the image analysis tool Optimas (Optimas Corp., Wash.) for filtering and quantitation. A median filter was run, followed by Wallis filtering with a 5×5 grid size. A 5×5 averaging filter was then applied three times. For colony detection, a threshold intensity was set such that only active colonies were highlighted. Using Optimas, we calculated intensity, area, and circularity statistics for each colony. To determine the fraction of wild-type activity retained by each colony, the difference of the mean colony intensity and the threshold intensity is divided by the difference of the average wild-type mean colony intensity and the threshold intensity.

Error-prone PCR of Gene Coding for the Large Subunit of Toluene Dioxygenase and Library Construction

Two primers (5′-CGGAATTCTAGGAAACAGACCATG-3′ [SEQ ID NO: 1] and 5′ -CCGGATCCAACCTGGGTCGAAGTCAAATG-3′ [SEQ ID NO: 2]) were used to amplify the gene encoding the large subunit of toluene dioxygenase under error-prone conditions. A reaction volume of 100 μL contained: 133 pg of pJMJ8-like plasmid DNA, 40 pmoles of each primer, 1×Taq buffer, 0.2 μmoles of each dNTP, 0.7 μmoles of MgCl₂, 60 nmoles of MnCl₂, and 2.5U of AmpliTaq DNA polymerase. PCR was carried out in a MJ Research PTC-200 thermal cycler (Watertown, Mass.) under the following conditions: 3 minutes at 94° C., 30 cycles of (30 seconds at 94° C., 30 seconds at 50° C., 1 minute at 72° C.), and 3 minutes at 72° C. PCR product was digested with EcoRI and BamHI and ligated into a similarly digested vector using T4 DNA ligase. Restriction enzymes and ligase were used according to manufacturer's instructions. 3 μL of ligation product were mixed with 100 μL of BL21 (DE3) competent cells and transformed using the supplier's protocol.

Results and Discussion

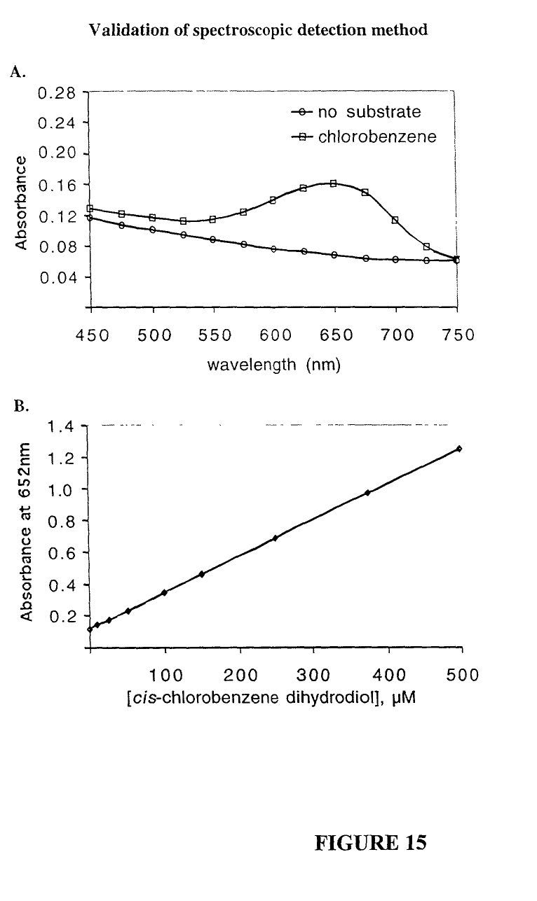

Validation of spectrophotometric detection of cis-dihydrodiols. FIG. 15A shows the visible absorbance spectrum for the colored products resulting from acidification of cis-chlorobenzene dihydrodiol followed by coupling with Gibbs reagent. There was a linear relation between absorbance and cis-chlorobenzene dihydrodiol concentration shown in FIG. 15B, that shows the sample absorbance (up to at least 1.2) accurately reflects the relative amount of d1hydrodiol produced by the biocatalyst.

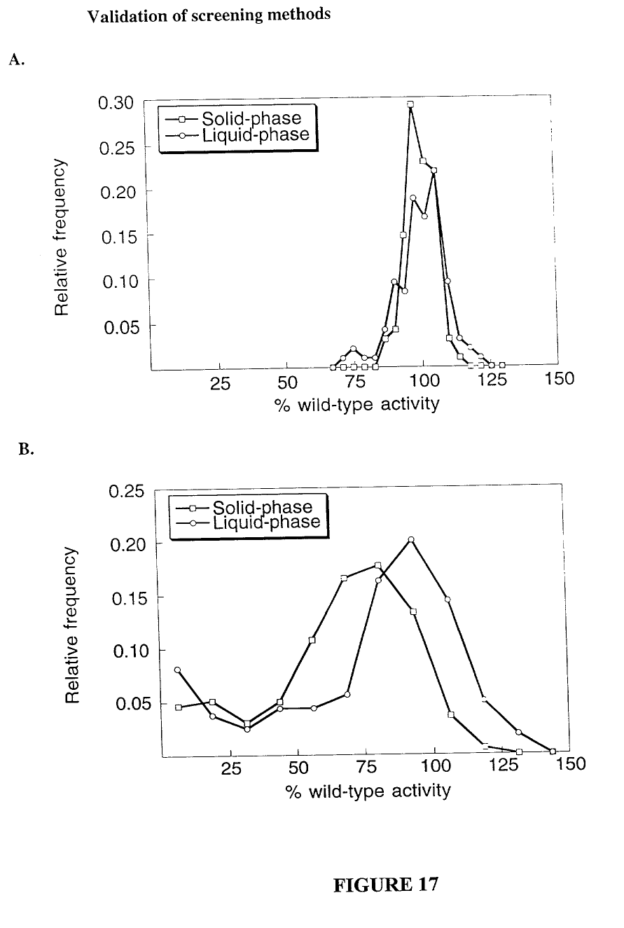

Estimation of error associated with high-throughput measurements. The liquid- and solid-phase methods described above are different, and can be expected to have different amounts of uncertainty associated with the measurements. The liquid-phase chemistry (shown in FIG. 14B) is executed in 96-well plates, while the solid-phase approach (shown in FIG. 14C requires coexpression of dehydrogenase and can be applied to more than 500 colonies on a single nitrocellulose membrane. To compare the uncertainty associated with the activity measurements of these two methods, we transformed BL21 (DE3) cells with either wild-type pJMJ8 or pJMJ2, and 96 individual clones were screened for activity toward chlorobenzene using the liquid- and solid-phase methods, respectively. The distributions of activity measurements for these two experiments are shown in FIG. 17A. The standard deviation of measured activity toward chlorobenzene was 9.0% of the activity of wild-type with the liquid-phase assay and only 5.3% of the activity of wild-type with the solid-phase method.

Comparative evaluation of liquid- and solid-phase methods. As an additional validation of the solid-phase approach, a mutant library of toluene dioxygenase was screened using both methods. The gene for the large subunit of toluene dioxygenase was subjected to error-prone PCR and cloned into pJMJ2 and pJMJ8. One hundred sixty mutant pJMJ8 clones and 1899 mutant pJMJ2 clones were screened for activity toward chlorobenzene using the liquid- and solid-phase methods, respectively. Activity measurements are plotted in histogram form in FIG. 17B. Similar bulk library characteristics (fraction inactive clones, fraction with wild-type-like activity) were found using the two methods. We thus conclude that the relative activities of clones in a mutant library do not depend significantly on the cell-growth method (on agar or liquid medium) or the specific method of detection (FIG. 14).

The solid-phase method eliminates the time-consuming step of inoculating colonies into microtiter plates. This increases the throughput of the screen to about 10,000 clones/day, compared to about 1000 clones/day for the liquid-phase approach. Although the liquid-phase method is more amenable to robotic automation, it requires much more technician hands on time and has a higher consumables cost per clone screened. With the solid-phase method, improved clones are recovered from the original growth plate by visual comparison with the digital image. With the liquid-phase method, clones can be recovered from the well of the 96-well plate in which they were originally inoculated.

### Bibliography

Adam, H. K., et al. 1981. Estimation of ICI 35,868 ( Diprivan^{R}) in blood by high-performance liquid chromatography, following coupling with Gibbs' reagent. J. Chromatogr. 223, 232-237.

Amann, E., et. al. 1988. Tightly regulated tac promoter vectors useful for the expression of unfused and fused proteins in E. Coli. Gene 69:301-315.

Arisawa, A. 1999. Directed Evolution of Toluene Dioxygenase; Generation of Enzyme Which has Increased Activity Towards Chlorobenzene. Unpublished.

Baggi, T. R., et al. 1974. Spectrophotometric determination of piperidine in pharmaceutical preparations. J. Assoc. Off Anal. Chem. 57, 1144-1146.

Baggi, T. R., et al. 1976. Visualisation of opium alkaloids on TLC plates by Gibbs reagent spray. Forensic Sci. 8, 265-267.

Belal, F. 1984. Use of 2,6-dichloroquinone chloroimide for the spectrophotometric determination of halogenated derivatives of 8-hydroxyquinoline. Analyst 109, 615-618.

Boyd, D. R., et. al. 1991. Enzymatic and Chemical Syntheses of Cis-dihydrodiol Derivatives of Monocyclic Arenes. J. Chem. Soc., Chem. Commun. 22:1630-1632.

Boyd, D. R., et. al. 1998. Enzymatic and chemoenzymatic synthesis and stereochemical assignment of cis-dihydrodiol derivatives of monosubstituted benzenes. J. Chem. Soc. Perkin Trans. 1:1935-1943.

Boyd, J. A., et al. 1984. Evidence for a one-electron mechanism of 2-aminofluorene oxidation by prostaglandin H synthase and horseradish peroxidase. J. Biol. Chem. 259, 13885-13896.

Bruhlman, F., and Chen, W. 1998. Tuning biphenyl dioxygenase for extended substrate specificity. Biotech. Bioeng. 63(5):544-551.

Butler C. S., and Mason, J. R. 1997. Structure-function analysis of the bacterial aromatic ring-hydroxylating dioxygenases. Adv. Micr. Phys. 38:47-84.

Cammack, R. and J. R. Mason. 1992. The Electron-Transport Proteins of Hydroxylating Bacterial Dioxygenases. Annu. Rev. Microbiol. 46:277-305.

Cook, D. L. and Atkins, W. M. Biochemistry, 36, 10801 (1997).

Coop A., et al. 1995. Methylation of the enolates of Thevinone and some analogues. Tetrahedron 51, 9681-9698.

Dacre, J. C. 1971. Nonspecificity of the Gibbs reaction. Anal. Chem. 43, 589-591

Drew, S. W., et. al. 1999. Microbial Conversion of Indene to Indandiol: A Key intermediate in the Synthesis of CRIXIVAN. Metabolic Engineering 1:63-74.

Ensley, B. D. 1994. Biosynthesis of the Textile Dye Indigo by a Recombinant Bacterium. Chimia 48:491-492.

Faber, K. Biotransformations in Organic Chemistry, Springer-Verlag, Berlin, p. 214, 217(1997).

Flavin, M. T., et al. 1996. Synthesis, chromatographic resolution, and anti-human immunodeficiency virus activity of (±)—Calanolide A and its enantiomers. J. Med. Chem. 39, 1303-1313.

Frost, J. W. and J. Lievense. 1994. Prospects for biocataltic synthesis of aromatics in the 21st century. New J. Chem. 18:341-348.

Gibbs, H. D. 1927. J. Biol. Chem. 72, 649-664.

Gibson, D. T. and G. J. Zylstra. 1989. Toluene Degradation by Pseudomonas putida F1. J. of Bio. Chem. 264(25):14940-14946.

Gibson, D. T. and S. M. Resnick. 1996:1. Oxidation of 6,7-Dihydro-5H-Benzocyclo-heptene by Bacterial Strains Expressing Naphthalene Dioxygenase, Biphenyl Dioxygenase, and Toluene Dioxygenase Yields Homochiral Monol or Cis-diol Enantiomers as Major Products. Appl. Env. Micro. 62(4):1364-1368.

Gibson, D. T. and S. M. Resnick. 1996:2. Regio- and Stereospecific Oxidation of 9,10-Dihydroanthracene and 9,10-Dihydrophenanthrene by Naphthalene Dioxygenase. Appl. Env. Microbiol. 62(9):3355-3359.

Gibson, D. T. and S. M. Resnick. 1996:3. Diverse Reactions Catalyzed by Naphthalene Dioxygenase from Psuedomonas sp. strain NCIB 9816. J. of Ind. Microbiol. 17:438-457.

Gibson, D. T., et. al. 1970. Formation of (+)-cis-2,3-Dihydroxy-1-methylcyclohexa-4,6-diene from Toluene by Psuedomonas Putida. Biochemistry 9:1626-1630.

Gibson, D. T., et. al. 1989. Monohydroxylation of Phenol and 2,5-Dichlorophenol by Toluene Dioxygenase in Psuedomonas putida F1. Appl. Env. Micro. 55(10):2648-2652.

Gibson, D. T., et. al. 1992. Oxidation of Nitrotoluenes by Toluene Dioxygenase: Evidence for a Monooxygenase Reaction. Appl. Env. Micro. 58(8):2643-2648.

Gilbert, S. C., et al. 1998. Isolation of a unique benzothiophene-desulphurizing bacterium, Gordona sp. strain 213E (NCIMB 40816), and characterization of the desulphurization pathway. Microbiology 144, 2545-2553.

Gibson, D. T., and Parales, R. E. 2000. Aromatic hydrocarbon dioxygenases in environmental biotechnology. Curr Opin Biotechnol. 11:236-243.

Hallit, Z., et al. 1981. A new histochemical method for demonstration of sulfhydryl groups. Microsc. Acta 84, 1-7.

Harfoush, A. A. 1986. Medium effect on the reaction between Gibb's reagent and thiosulphate. Z. phys. Chemie, Leipzig 267, 682-688.

Harrop, A. j., et al. 1992. Production of naphthalene-cis-glycol by Pseudomonas putida in the presence of organic solvents. Enz. Micr. Tech. 72:649-664.

Hudlicky, T., et. al. 1996:1. Toluene Dioxygenase-Mediated cis-Dihydroxylation of Aromatics in Enantioselective Synthesis. J. Am. Chem. Soc. 118:10752-10765.

Hudlicky, T., et. al. 1996:2. Current status and future perspectives of cyclohexadiene-cis-diols in organic synthesis. Chem. Commun. 17:1993-2000.

Jenkins, R. O., et. al. 1986. Production of toluene cis-glycol by Pseudomonas putida in glucose fed-batch culture. Biotech. Bioeng. 29:873-883.

Johnston, J. B., et al. 1987. Production of substituted catechols from substituted benzenes by a Pseudomanas sp. Enzyme Microb. Technol. 9, 706-708.

Josephy, P. D. 1984. Reaction of Gibbs reagent with para-substituted phenols. Anal. Chem. 56, 813-814.

Josephy, P. D., et al. 1984. Reaction of Gibbs reagent (2,6-dichlorobenzoquinone 4-chloroimine) with the antioxidant BHA (3-tert.-butyl 4-hydroxyanisole): isolation and identification of the major product. J. Chromatogr. 294, 375-379.

Kayser, K. J., et al. 1993. Utilization of organosulphur compounds by axenic and mixed cultures of Rhodococcus rhodochrous IGTS8. J. Gen. Microbiol. 139, 3123-3129.

Kobylianskii, A. G., et al. 1994. Determination of free and bound phenol in urine during mass screening of the populace. Vopr. Med. Khim. 40, 40-46.

Kumamaru, T., et al. 1998. Enhanced degradation of PCB's by directed evolution of biphenyl dioxygenase. Nat. Biotech. 16:663-666.

Lange C. C., and Wackett, L. P. 1997. Oxidation of aliphatic olefins by toluene dioxygenase: enzyme rates and product identification. J. Bac. 179:3858-3865.

Mason, J. R., and C. S. Butler. 1997. Structure-Function Analysis of the Bacterial Ring-Hydroxylating Dioxygenases. Adv. Microbial Phys. 38:47-84.

McAllister, R. A. 1951. A colorimetric method for the determination of 1-methyl-2-mercaptoimidazole. J. Pharm., Lond. 3, 506-510.

Miller, P., et al. 1990. Dichlorobenzoquinone chloroimine colorimetry of uric acid in urine. Clin. Chem. 36, 668-669.

Miyazaki, K., et al. 2000. Directed evolution study of temperature adaptation in a psychrophilic enzyme. J. Mol. Biol. 297:1015-1026.

Mondello, F., et. al. 1997. Identification and Modification of Biphenyl Dioxygenase Sequences That Determine the Specificity of Polychlorinated Biphenyl Degradation. Appl. Env. Microbiol. 63(8):3096-3103.

Mondello, F. J. 1988. Cloning and Expression in E. coli of Psuedomonas sp. strain LB400 Genes Encoding Biphenyl Degradation. J. Bac. 171(3):1725-1732.

Moore, J. C., and Arnold, F. H. 1996. Directed evolution of a para-nitrobenzyl esterase for aqueous-organic solvents. Nat. Biotech. 14:458-467.

Orlewski, P., et al. 1982. Usefulness of a modified screening method for the detection of phenol in the saliva in evaluating occupational exposure to this compound. Med. Pr. 33, 215-221.

Pallagi, I., et al. 1994. Mechanism of the Gibbs reaction. 3. Indophenol formation via radical electrophilic aromatic substitution (S_{RE}Ar) on phenols. J. Org. Chem. 59, 6543-6557.

Pallagi, I., et al. 1999. Mechanism of the Gibbs reaction. 4. Indophenol formation via N-chlorobenzoquinone imine radical anions. The aza-S_{RN}3 chain reaction mechanism. Chain initiation with 1,4-benzoquinones and cyanide ion. J. Org. Chem. 64, 6530-6540.

Panadero, S., et al. 1993. Kinetic determination of Dicoumarol on grain by using stopped-flow mixing methodology. Intern. J. Anal. Chem. 50, 45-51.

Pieper, D. H., et. al. 1997. Metabolism of Chlorotoluenes by Burkholderia sp. Strain PS12 and Toluene Dioxygenase of Pseudomonas putida F1: Evidence for Monooxygenation by Toluene and Chlorobenzene Dioxygenases. App. Env. Micro. 63(5):1974-1979.

Pieper, D. H., et. al. 1998. Identification of Chlorobenzene Dioxygenase Sequence Elements Involved in Dechlorination of 1,2,4,5-Tetrachlorobenzene. J. Bac. 180(21):5520-5528.

Quintana, M. G., et. al. 1997. Colorimetric method for a rapid detection of oxygenated aromatic biotransformation products. Biotechnol. Tech. 11:585-587.

Reider, P. J. 1997. Advances in AIDS chemotherapy: The asymmetric synthesis of CRIXIVAN. Chimia. 51:306-308.

Sambrook, J., E. F. Fritsch, and T. Maniatis. 1989. Molecular Cloning. Cold Spring Harbor Laboratory Press, United States of America.

Searle, C. E. 1955. Colour reactions of 2:6-dichloroquinone chloroimide with derivatives of glyoxaline-2-thiol. J. Appl. Chem. 5, 313-316.

Sheldrake, G. N. 1992. “Biologically Derived Arene Cis-dihydrodiols as Synthetic Building Blocks,” In: Chirality in Industry. Collins, Sheldrake, and Crosby (Eds.), pp. 127-166. John Wiley & Sons Ltd., New York.

Short, J. Nature Biotechnol. 15, 1322 (1997).

Singh, J., et al. 1974. A method for α-L-iduronidase assay. FEBS Lett. 45, 248-251.

Suzuki, Y., et al. 1992. Formation of β-galactosyl compounds of pyridoxine in growing culture of Sporobolomyces singularis. Biochim. Biophs. Acta 1116, 67-71.

Tadera, K., et al. 1988. Isolation and structural elucidation of three new pyridoxine-glycosides in rice bran. J. Nutr. Sci. Vitaminol. 34, 167-177.

Uemura, T., et al. 1988. Isolation, structure, and properties of the β-Carboline formed from 5-hydroxytryptamine by the superoxide anion-generating system. J. Neurochem. 51, 710-717.

Umezawa, H., et al. 1987. Novel physiologically active substance Benadrostin and production thereof. JP62281871A.

Velosy, G. A. 1985. New method for the continuous determination fo the catalytic activity of pancreatic carboxyesterase. J. Clin. Chem. Clin. Biochem. 23, 887-889.

Vinson, J. A., et al. 1977. Sensitive thin-layer chromatographic method for urine screening of barbiturates. J. Chromatogr. 140, 71-76.

Wackett, L. P., et. al. 1990. The application of toluene dioxygenase in the synthesis of acetylene-terminated resins. App. Microbiol. Biotech. 34:316-321.

Wackett, L. P. 1995. Recruitment of co-metabolic enzymes for environmental detoxification of organohalides. Environ. Health Perspect. 103:45-48.

Wackett, L. P., et. al. 1997. Oxidation of Aliphatic Olefins by Toluene Dioxygenase: Enzyme Rates and Product Identification. J. Bac. 179(12):3858-3865.

Wahbi, L. P., et. al. 1996. Construction and use of recombinant E. coli strains for the synthesis of toluene cis-glycol. Enzyme and Microbial Technology 19:297-306.

Wendeborn, S., et al. 1998. Polymer Bound 3,5-Cyclohexadiene-1,2-diols as Core Structures for the Development of Small Molecule Libraries. Synlett. 8:865-868.

Wilkinson, D., et al. 1996. Choice of bacterial host for the naphthalene dioxygenase bioconversion. J. Ind. Micr. 16:274-279.

Wojtaszek P., et al. 1992. A simple method for visualization of phenolics exudation by roots of intact lupin plants; The effect of nitrate and pH. Acta Biochim. Pol. 39, 307-316.

Woodley, J. M., et. al. 1992. Production of naphthalene-cis-glycol by Psuedomonas putida in the presence of organic solvents. Enzyme Microb. Technol. 14:725-730.

Woodley, J. M., et. al. 1996. Choice of microbial host for the napthalene dioxygenase bioconversion. Journal of Industrial Microbiology 16:274-279.

Zhao, H., et al. 1999. Methods for optimizing industrial enzymes by directed evolution. in Mannual of Industrial Microbiology and Biotechnology, 2nd Ed. (Demain, A. L., & Davies J. E. , Eds), 597-604 ASM press, Washington, D.C.

Zylstra G. J., and Gibson, D. T. 1989. Phenol tests III, the indophenol test. J. Biol. Chem. 264:14940-14946.

Patent Literature

U.S. Pat. No. 5,605,793

U.S. Pat. No. 5,741,691

U.S. Pat. No. 5,811,238

WO Publication No. 98/42832

We claim:

1. A method for detecting an oxidation enzyme comprising the steps of:
(a) contacting a test enzyme with a substrate and an oxygen donor to promote the formation of a cis-dihydrodiol from the substrate and the oxygen donor;
(b) subjecting the cis-hydrodiol to acidic conditions to promote the formation of a phenol from the cis-dihydrodiol;
(c) contacting the phenol with 2,6-dichloroquinone-4-chloroimide (Gibbs reagent) under neutral conditions to promote the formation of a detectable composition; and
(d) testing for the detectable composition, wherein the presence of the detectable composition indicates that the test enzyme is an oxidation enzyme.

2. The method of claim 1, wherein the oxidation enzyme is selected from the group consisting of a monooxygenase enzyme and a dioxygenase enzyme.

3. The method of claim 1, wherein the oxidation enzyme is selected from the group consisting of toluene dioxygenase, biphenyl dioxygenase, naphthalene dioxygenase, methane monooxygenase, chloroperoxidase, cytochrome P450, phenol hydroxylase, dehalogenase, and microperoxidase.

4. The method of claim 1, wherein the test enzyme is a mutant enzyme or a wild-type enzyme.

5. The method of claim 1, wherein the substrate is selected from the group consisting of an aromatic hydrocarbon and a halogenated ethylene.

6. The method of claim 1, wherein the substrate is selected from benzene, toluene, t-butylbenzene, 1,2,4-trimethylbenzene, flurorbenzene, chlorobenzene, bromobenzene, iodobenzene, benzoic acid, p-methoxybenzoic acid, 2-napthoic acid, benzamide, pyridine, and 4-picoline.

7. The method of claim 1, wherein the oxygen donor is selected from the group consisting of molecular oxygen and a peroxide.

8. The method of claim 1, wherein the acidic conditions are provided by lowering the pH to about 2.5.

9. The method of claim 1, wherein the neutral conditions are provided by increasing the pH to within the range from about 7 to about 9.

10. The method of claim 1, wherein the test enzyme is expressed in a host cell, and the substrate and oxygen donor contacted with the host cell.

11. The method of claim 10, wherein the host cell comprises a plasmid comprising a gene encoding the test enzyme.

12. The method of claim 10, wherein the host cell is attached to a solid support.

13. The method of claim 12, wherein the solid support is selected from the group consisting of agar and a membrane.

14. The method of claim 12, wherein colonies of multiple host cells are spread on the solid support.

15. The method of claim 1, wherein the detectable composition is a colored product detectable by visual inspection, spectrometry, or digital imaging.

16. A method for detecting an oxidation enzyme comprising the steps of:
(a) contacting an test enzyme with a substrate to promote the formation of a cis-dihydrodiol from the substrate;
(b) contacting the cis-hydrodiol with cis-dihydrodiol dehydrogenase to promote the formation of a catechole from the cis-hydrodiol;
(c) contacting the catechol with 2,6-dichloroquinone-4-chloroimide (Gibbs reagent) to promote the formation of a detectable composition; and
(d) testing for the detectable composition, wherein the presence of the detectable composition indicates that the test enzyme is an oxidation enzyme.

17. The method of claim 16, wherein the oxidation enzyme is selected from the group consisting of a monooxygenase enzyme and a dioxygenase enzyme.

18. The method of claim 16, wherein the oxidation enzyme is selected from the group consisting of toluene dioxygenase, biphenyl dioxygenase, naphthalene dioxygenase, methane monooxygenase, chloroperoxidase, cytochrome P450, phenol hydroxylase, dehalogenase, and microperoxidase.

19. The method of claim 16, wherein the test enzyme is a mutant enzyme or a wild-type enzyme.

20. The method of claim 16, wherein the substrate is selected from the group consisting of an aromatic hydrocarbon and a halogenated ethylene.

21. The method of claim 16, wherein the substrate is selected from benzene, toluene, t-butylbenzene, 1,2,4-trimethylbenzene, flurorbenzene, chlorobenzene, bromobenzene, iodobenzene, benzoic acid, p-methoxybenzoic acid, 2-napthoic acid, benzamide, pyridine, and 4-picoline

22. The method of claim 16, wherein the oxygen donor is selected from the group consisting of molecular oxygen and a peroxide.

23. The method of claim 16, wherein the test enzyme is expressed in a host cell, and the substrate and oxygen donor are contacted with the host cell.

24. The method of claim 23, wherein the host cell also expresses cis-dihydrodiol dehydrogenase.

25. The method of claim 24, wherein the host cell comprises a plasmid comprising genes encoding the test enzyme and cis-dihydrodiol dehydrogenase.

26. The method of claim 23, wherein the host cell is attached to a solid support.

27. The method of claim 26, wherein the solid support is selected from the group consisting of agar and a membrane.

28. The method of claim 26, wherein colonies of multiple host cells are spread on the solid support.

29. The method of claim 16, further comprising contacting cis-dihydrodiol dehydrogenase with a coenzyme.

30. The method of claim 28, wherein the coenzyme is NAD⁺.

31. The method of claim 16, wherein the detectable composition is a colored product detectable by visual inspection, spectrometry, or digital imaging.

32. A method for detecting an oxidation enzyme comprising the steps of:
(a) contacting an test enzyme with a substrate to promote the formation of a product from the substrate;
(b) contacting the product with an agent to promote the formation of a modified product, wherein the modified product is selected from the group consisting of a phenol and a catechol;
(c) contacting the modified product with 2,6-dichloroquinone-4-chloroimide (Gibbs reagent) to promote the formation of a detectable composition; and
(d) testing for the detectable composition, wherein the presence of the detectable composition indicates that the test enzyme is an oxidation enzyme.

33. The method of claim 32, wherein the oxidation enzyme is selected from the group consisting of a monooxygenase enzyme and a dioxygenase enzyme.

34. The method of claim 32, wherein the oxidation enzyme is selected from the group consisting of toluene dioxygenase, biphenyl dioxygenase, naphthalene dioxygenase, methane monooxygenase, chloroperoxidase, cytochrome P450, phenol hydroxylase, dehalogenase, and microperoxidase.

35. The method of claim 32, wherein the test enzyme is a mutant enzyme or a wild-type enzyme.

36. The method of claim 32, wherein the test enzyme is expressed in a host cell, and the substrate and oxygen donor are contacted with the host cell.

37. The method of claim 32, wherein the substrate is selected from the group consisting of an aromatic hydrocarbon and a halogenated ethylene.

38. The method of claim 32, wherein the substrate is selected from benzene, toluene, t-butylbenzene, 1,2,4-trimethylbenzene, flurorbenzene, chlorobenzene, bromobenzene, iodobenzene, benzoic acid, p-methoxybenzoic acid, 2-napthoic acid, benzamide, pyridine, and 4-picoline

39. The method of claim 32, wherein the oxygen donor is selected from the group consisting of molecular oxygen and a peroxide.

40. The method of claim 32, wherein the product is selected from a cis-dihydridiol, an alkylated benzene, a halogenated benzene, and a carboxylated benzene.

41. The method of claim 40, wherein the product is anthranilic acid

42. The method of claim 32, wherein the agent is an acid.

43. The method of claim 32, wherein the agent is an enzyme.

44. The method of claim 42, wherein the enzyme is a second oxidation enzyme.

45. The method of claim 43, wherein the product is a cis-dihydrodiol and the second oxidation enzyme is a cis-dihydrodiol dehydrogenase.

46. The method of claim 43, wherein the product is a halogenated benzene, and the second oxidation enzyme is a dehalogenase.

47. The method of claim 43, wherein the product is selected from the group consisting of alkylated and carboxylated benzene, an the second oxidation enzyme is selected from the group consisting of cytochrome P450 and a peroxidase.

48. The method of claim 43, wherein the product is anthranilic acid, and the second oxidation enzyme is anthranilite monooxygenase.

49. The method of claim 36, wherein the host cell also expresses a second oxidation enzyme, which second oxidation enzyme promotes the formation of a modified product.

50. The method of claim 49, wherein the host cell comprises a plasmid comprising genes encoding the test enzyme and the second oxidation enzyme.

51. The method of claim 49, wherein the product is a cis-dihydrodiol and the second oxidation enzyme is a cis-dihydrodiol dehydrogenase.

52. The method of claim 51, further comprising contacting the cis-dehydydiol dehydrogenase with NAD⁺.

53. The method of claim 36, wherein the host cell is attached to a solid support.

54. The method of claim 53, wherein the solid support is selected from the group consisting of agar and a membrane.

55. The method of claim 53, wherein colonies of multiple host cells are spread on the solid support.

56. The method of claim 32, wherein the detectable composition is a colored product detectable by visual inspection, spectrometry, or digital imaging.

57. A method for detecting an oxidation enzyme comprising the steps of:
(a) contacting test enzyme with a substrate to promote the formation of a phenol ether, wherein the hydroxyl-group is attached to the aromatic part of the phenol ether;
(b) contacting the phenol ether with 2,6-dichloroquinone-4-chloroimide (Gibbs reagent) to promote the formation of a detectable composition; and
(c) testing for the detectable composition, wherein the presence of the detectable composition indicates that the test enzyme is an oxidation enzyme.

58. The method of claim 57, wherein the oxidation enzyme is cytochrome P450.

59. The method of claim 57, wherein the test enzyme is a mutant enzyme or a wild-type enzyme.

60. A method for detecting an oxidation enzyme comprising the steps of:
(a) contacting a test enzyme with a substrate to promote the formation of a phenol ether, wherein the hydroxyl-group is attached to the ether part of the phenol ether;
(b) allowing the phenol ether to dissociate into an aldehyde and a phenol;
(c) contacting the phenol with 2,6-dichloroquinone-4-chloroimide (Gibbs reagent) to promote the formation of a detectable composition; and
(d) testing for the detectable composition, wherein the presence of the detectable composition indicates that the test enzyme is an oxidation enzyme.

61. The method of claim 60, wherein the oxidation enzyme is cytochrome P450.

62. The method of claim 60, wherein the test enzyme is a mutant enzyme or a wild-type enzyme.
